(19) United States
(12) Patent Application Publication
(10) Pub. No.: US 2024/0011030 A1
(43) Pub. Date: Jan. 11, 2024

(54) TREATMENTS FOR RETINAL DEGENERATIVE DISEASES

(71) Applicant: Novartis AG, Basel (CH)

(72) Inventors: Yi BAO, West Roxbury, MA (US); Qian HUANG, Somerville, MA (US); Joshua SCHUSTAK, Cambridge, MA (US); Michael Zbigniew TWAROG, Cambridge, MA (US)

(21) Appl. No.: 18/020,329

(22) PCT Filed: Aug. 9, 2021
(86) PCT No.: PCT/IB2021/057323

§ 371 Date: Feb. 8, 2023

Related U.S. Application Data
(60) Provisional application No. 63/063,877, filed on Aug. 10, 2020.

(51) Int. Cl. C12N 15/113; C12N 15/86; C12N 9/22; A61K 31/407; A61K 9/00

(52) U.S. Cl. CPC C12N 15/113; C12N 15/86; C12N 9/22; A61K 31/407; A61K 9/0048; C12N 2310/20; C12N 2310/14; C12N 2740/15043

(57) ABSTRACT

Methods for treating or improving a retinal degenerative disease or condition in a subject in need thereof are provided. In some embodiments described herein, the methods comprise administering to the subject a therapeutically effective amount of a retinoic acid-inducible gene I (RIG-I) inhibitor.

### CROSS-REFERENCE TO RELATED APPLICATIONS

This application claims the benefit of and priority to U.S. Patent Application. No. 63/063,877, filed Aug. 10, 2020, which is herein incorporated by reference in its entirety, for all purposes.

### SEQUENCE LISTING

The instant application contains a Sequence Listing which has been submitted electronically in ASCII format and is hereby incorporated by reference in its entirety. Said ASCII copy, created on Aug. 5, 2021, is named PAT058930-WO-PCT_SL.txt and is 21,554 bytes in size.

### FIELD OF THE INVENTION

The present disclosure relates to methods for treating or preventing a retinal degenerative disease or condition in a subject by administering a retinoic acid-inducible gene I (RIG-I) inhibitor. The present disclosure also relates to methods for improving vision in a subject through administration of a RIG-I inhibitor.

### BACKGROUND

Age-related macular degeneration (AMD) is the most common cause of blindness among the elderly in the United States and worldwide (1). Although there are anti-angiogenic drugs for wet AMD, to date there are no Food and Drug Administration (FDA)-approved treatments for intermediate AMD or geographic atrophy (GA).

Retinal pigment epithelium (RPE) comprises unique epithelial cells essential for maintaining vision. RPE interacts with photoreceptors on its apical side and with Bruch's membrane and the choriocapillaris on its basal side. Damage to the RPE is associated with pathogenesis of AMD. Aging and other accumulated genetic and environmental risk factors may lead to RPE dysfunction, eventually resulting in cell death, a major pathology of AMD. However, the cellular mechanisms causing the RPE to degenerate are poorly understood.

Chronic inflammatory events, e.g., complement, Toll-Like Receptor (TLR), Nuclear Factor κ B (NFkB), inflammasome, and type I interferon (IFN) responses, may play a central role in the pathogenesis and development of AMD (2,3). However, the precise molecular nature of inflammatory mechanisms that promote AMD development remain unclear. The immune status of the chorioretinal interface is mainly regulated by the RPE, and inflammation may have both a physiological role in RPE degeneration and a pathological role in AMD development. The type I interferon (type I IFN) response may be a key regulatory pathway in genetic (e.g., A/J mouse) and abnormal metabolite accumulation (e.g., Alu-RNA)-induced rodent retinal degeneration models (4,5). However, the cell type(s) responsible for mediating type I IFN response and the mechanisms by which cells generate and respond to IFN in human patients are still not fully known. Moreover, the major inducers and sensors responsible for the type I IFN pathway in the retina remain unidentified.

Therefore, there remains a need to develop effective treatments for retinal disorders such as AMD and GA.

### SUMMARY

The current disclosure is based in part upon the finding that RIG-I unexpectedly plays a key role in nucleic acid-induced inflammation in cells of the retina, for example, retinal pigment epithelium cells. In particular, the current disclosure is based upon the finding that RIG-I plays a key role in mediating type I interferon (for example, interferon β) response in cells of the retina, for example, in retinal pigment epithelium cells. The current disclosure is also based upon the finding that inhibiting RIG-I can provide a method of treating a retinal degenerative disease or condition, for example, AMD or GA.

Thus, in one embodiment of the invention, disclosed herein is a method for treating a retinal degenerative disease or a condition in a subject in need thereof, the method comprising administering to the subject a therapeutically effective amount of a retinoic acid-inducible gene I (RIG-I) inhibitor or a pharmaceutical composition thereof. In some embodiments, the retinal degenerative disease or condition is age-related macular degeneration (AMD). In some embodiments, the AMD is early stage AMD. In some embodiments, the AMD is late-stage AMD. In some embodiments, the AMD is geographic atrophy (GA). In some embodiments, the AMD is wet AMD or dry AMD. In some embodiments, the retinal degenerative disease or condition is diabetic macular edema (DME). In some embodiments, the retinal degenerative disease is abnormal angiogenesis, choroidal neovascularization (CNV), retinal vascular permeability, retinal edema, diabetic retinopathy (for example, proliferative diabetic retinopathy), neovascular (exudative) age-related macular degeneration (AMD), including CNV associated with nAMD (neovascular AMD), sequela associated with retinal ischemia, Central Retinal Vein Occlusion (CRVO), or posterior segment neovascularization.

In another embodiment of the invention, disclosed herein is a method for reducing progression of a GA lesion (for example, progression of GA lesion enlargement) in a subject, the method comprising administering to the subject a therapeutically effective amount of a RIG-I inhibitor or a pharmaceutical composition thereof. In one embodiment, disclosed herein is a method for terminating progression of GA lesion enlargement in a subject, the method comprising administering to the subject a therapeutically effective amount of a RIG-I inhibitor or a pharmaceutical composition thereof. In one embodiment, disclosed herein is a method for treating (for example, reducing in size or eliminating) a GA lesion in a subject, the method comprising administering to the subject a therapeutically effective amount of a RIG-I inhibitor or a pharmaceutical composition thereof.

In yet another embodiment of the invention, disclosed herein is a method for improving vision in a subject, the method comprising administering to the subject a therapeutically effective amount of a RIG-I inhibitor or a pharmaceutical composition thereof. For example, in some embodiments, disclosed is a method for improving visual acuity, retinal focus, or central vision the method comprising administering to the subject a therapeutically effective amount of a RIG-I inhibitor or a pharmaceutical composition thereof.

In yet another embodiment of the invention, disclosed herein is a method for inhibiting RPE degeneration in an eye of a subject, the method comprising administering to the subject a therapeutically effective amount of a RIG-I inhibitor or a pharmaceutical composition thereof.

In yet another embodiment of the invention, disclosed herein is a method for inhibiting transepithelial resistance impairment of RPE in an eye of a subject, the method comprising administering to the subject a therapeutically effective amount of a RIG-I inhibitor or a pharmaceutical composition thereof.

In methods described herein, the RIG-I inhibitor can be a small molecule chemical compound, an antibody (for example, a RIG-I specific antibody), an antigen-binding fragment of a RIG-I specific antibody, a nucleic acid molecule, a peptide, or a derivative thereof. For example, in an embodiment described herein, the RIG-I inhibitor can be a small molecule chemical compound, an antibody, an antigen-binding fragment of an antibody, a nucleic acid molecule, or a peptide that inhibits RIG-I activity or RIG-I expression, when administered in a therapeutically effective amount. For example, in some embodiments, a RIG-I inhibitor described herein can inhibit RIG-I gene transcription, RIG-I mRNA translation, RIG-I protein expression, or RIG-I post-translational protein modification. In some embodiments, a RIG-I inhibitor described herein can inhibit RIG-I enzymatic activity (e.g., RIG-I RNA helicase activity), RIG-I nucleic acid sensing, RIG-I nucleic acid binding, RIG-I signaling transduction, or RIG-I induction of interferon expression.

In some embodiments of the invention, administering a RIG-I inhibitor comprises administering a pharmaceutical formulation comprising a RIG-I inhibitor and a pharmaceutically acceptable excipient. Thus, in some embodiments, disclosed herein is a method of treating a retinal degenerative disease or condition in a subject in need thereof; a method for reducing the progression of GA lesion enlargement in a subject; a method for improving vision in a subject; a method for inhibiting RPE degeneration in an eye of a subject; and a method for inhibiting transepithelial resistance impairment of RPE in an eye of a subject. In any of the aforementioned embodiments, the method can include administering to the subject a pharmaceutical composition comprising a therapeutically effective amount of a RIG-I inhibitor and a pharmaceutically acceptable excipient.

In an embodiment of the invention described herein, the RIG-I inhibitor can be a siRNA, a miRNA, a shRNA, a ribozyme, a morpholino, an antisense RNA, a triple helix forming molecule, or a sgRNA. For example, in an embodiment described herein, the RIG-I inhibitor is a siRNA, a miRNA, a shRNA, a ribozyme, a morpholino, an antisense RNA, a triple helix forming molecule, or a sgRNA that inhibits RIG-I expression. In some embodiments described herein, the RIG-I inhibitor comprises a siRNA, a miRNA, a shRNA, a ribozyme, a morpholino, an antisense RNA, a triple helix forming molecule, or a sgRNA that is complementary to a portion of a RIG-I nucleic acid sequence. In some embodiments described herein, the RIG-I inhibitor comprises a siRNA, a miRNA, a shRNA, a ribozyme, a morpholino, an antisense RNA, a triple helix forming molecule, or a sgRNA, wherein a portion of the nucleic acid sequence of the RIG-I inhibitor is complementary to a portion of a RIG-I nucleic acid sequence.

In an embodiment of the invention described herein, the RIG-I inhibitor comprises a clustered regularly interspaced short palindromic repeats (CRISPR) system. In some embodiments, the CRISPR system is effective to inhibit RIG-I gene expression. In some embodiments, the CRISPR system is effective to modify a RIG-I gene sequence. For example, in an embodiment described herein the RIG-I inhibitor comprises a RIG-I CRISPRi system. Also in an embodiment described herein, the RIG-I inhibitor comprises a RIG-I CRISPR-Cas9 system. In embodiments wherein the RIG-I inhibitor comprises a RIG-I CRISPR system, the RIG-I CRISPR system can comprise a sgRNA, for example, a sgRNA that is complementary to a portion of a RIG-I gene sequence. In a particular embodiment, the RIG-I CRISPR-Cas9 system comprises an sgRNA comprising the sequence 5′-ACTCACCCTCCCTAAACCAG-3′ (SEQ ID NO:1), or a pharmaceutically acceptable salt thereof, or a derivative thereof.

In some embodiments of the invention described herein, the RIG-I inhibitor comprises a RIG-I siRNA (for example, a siRNA that is effective to bind to a portion of a RIG-I mRNA sequence). In some embodiments, upon binding to a RIG-I mRNA sequence, the RIG-I siRNA inhibits protein translation of the RIG-I mRNA sequence. In some embodiments, upon binding to a RIG-I mRNA sequence, the RIG-I siRNA mediates degradation of the RIG-I mRNA sequence (for example, RNA-Induced Silencing Complex (RISC)-mediated degradation of the RIG-I mRNA sequence). In some embodiments, the RIG-I siRNA comprises a nucleotide sequence selected from the group consisting of: 5′-AAGGGAACGATTCCATCACTA-3′ (SEQ ID NO:2), 5′-TTCTACAGATTTGCTCTACTA-3′ (SEQ ID NO:3), 5′-CTCCTCCTACCCGGCTTTAAA-3′ (SEQ ID NO:4), and 5′-CAGAATTATCCCAACCGATAT-3′ (SEQ ID NO:5), or a pharmaceutically acceptable salt thereof.

In some embodiments of the invention, the disclosed methods comprise a step of administering a RIG-I inhibitor or pharmaceutical composition thereof to a patient in need of treatment, wherein the RIG-I inhibitor comprises a peptide or a nucleic acid molecule encoding the peptide. For example, in some embodiments of the invention, the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof. In a particular embodiment described herein, the RIG-I inhibitor comprises the peptide sequence GNRDTLWHLFNTLQRRPGWVEYFI (SEQ ID NO:6), or a derivative thereof; or a nucleic acid molecule encoding the peptide of SEQ ID NO:6, or a derivative thereof.

In some embodiments of the invention, the RIG-I inhibitor further comprises a vector. For example, in embodiments described herein wherein the RIG-I inhibitor comprises a nucleic acid sequence encoding a RIG-I inhibitor (for example, a nucleic acid sequence encoding: an antibody or a portion of an antibody (for example, an antibody heavy chain or an antibody light chain), an antigen-binding fragment of a RIG-I specific antibody or a portion thereof, a nucleic acid molecule (for example, a shRNA or a sgRNA), a peptide (for example, a peptide of SEQ ID NO:6), or a CRISPR system or a portion thereof), the RIG-I inhibitor can further comprise a vector. In some embodiments, the vector is a viral vector. For example, in some embodiments the viral vector is a lentiviral vector. In some embodiments, the vector is an adenoviral vector, an adeno-associated virus (AAV) vector, a retroviral vector, or a derivative thereof.

In some embodiments of the invention, the RIG-I inhibitor comprises a small molecule compound. For example, in some embodiments, the RIG-I inhibitor is a small molecule compound selected from the group consisting of:

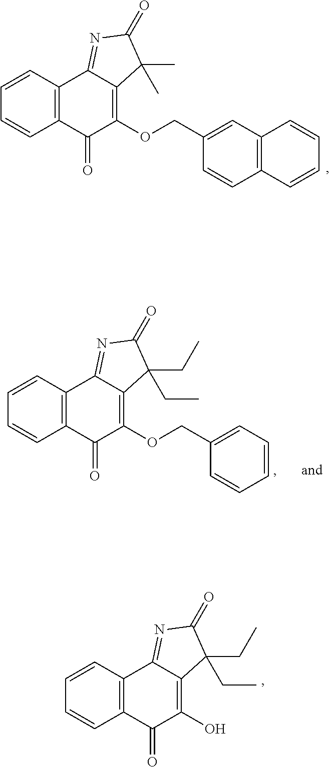 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]

or a pharmaceutically acceptable salt thereof.

In some embodiments of the invention, described herein is a method wherein the method comprises administering a RIG-I inhibitor or a pharmaceutical composition comprising a RIG-I inhibitor to a subject, for example, a subject in need thereof. In embodiments described herein, the administering can be by any route considered acceptable to those of skill in the art. For example, in embodiments described herein, administration of the RIG-I inhibitor can be, but is not limited to, oral, transdermal, topical, parenteral, injection, or inhalation routes. In some embodiments of the invention, the administering is locally to an eye of a subject. Thus, in some embodiments, the administering is subconjunctival, intravitreal, periocular, retrobulbar, or intracameral. In some embodiments of the invention, the administering comprises contacting an ocular cell of the subject with the RIG-I inhibitor. For example, in some embodiments, the administering comprises contacting a retinal cell of the subject, for example, a retinal pigment epithelial cell, with the RIG-I inhibitor.

In some embodiments of the invention described herein, administering of a therapeutically effective amount of a RIG-I inhibitor or a pharmaceutical composition thereof is effective to produce a specific biological outcome. For example, in some embodiments, administering a therapeutically effective amount of a RIG-I inhibitor reduces expression of a specific gene. In some embodiments, the administering is effective to reduce gene expression in an ocular cell of a subject. For example, in some embodiments, the administering is effective to reduce expression of an interferon stimulated gene (ISG) in an ocular cell of the subject. In some embodiments, the administering is effective to reduce expression of ISG15. In some embodiments, the administering is effective to reduce RIG-I protein level in an ocular cell of the subject. In some embodiments, the administering is effective to reduce RIG-I mRNA level in an ocular cell of the subject.

Additionally, in some embodiments of the invention, administering a therapeutically effective amount of a RIG-I inhibitor or a pharmaceutical composition thereof inhibits a particular cellular response. For example, in some embodiments, the administering is effective to reduce an IFN response in an ocular cell of a subject. In some embodiments, the IFN response is a type I IFN response. Thus, in some embodiments, the administering is effective to reduce expression of a type I interferon in an ocular cell of the subject. In some embodiments, the administering is effective to reduce secretion of a type I interferon in an ocular cell of the subject. In some embodiments, the type I interferon is IFNβ.

In some embodiments of the invention, administering a therapeutically effective amount of a RIG-I inhibitor to a subject is effective to inhibit nucleic acid-induced inflammation in an ocular cell of the subject. For example, in some embodiments of the invention, administering a therapeutically effective amount of a RIG-I inhibitor to a subject is effective to inhibit RNA-induced inflammation in an ocular cell of the subject. In some embodiments, the inflammation is induced by an intracellular source of RNA, for example, mitochondrial RNA, for example mitochondrial double-stranded RNA. Also disclosed herein is a method of inhibiting nucleic acid-induced (for example, RNA-induced) inflammation in a subject, wherein the method includes administering a RIG-I inhibitor to a subject in need of treatment. In some embodiments, the method of inhibiting nucleic acid-induced inflammation is effective to inhibit nucleic acid-induced inflammation in an eye of a patient. In some embodiments, the method of inhibiting nucleic acid-induced inflammation is effective to inhibit nucleic acid-induced inflammation in an ocular cell of a patient. In some embodiments, the inflammation in an eye of a patient is induced by an intracellular source of RNA, for example, mitochondrial RNA, for example mitochondrial double-stranded RNA. In some embodiments, the inflammation in an ocular cell of a patient is induced by an intracellular source of RNA, for example, mitochondrial RNA, for example mitochondrial double-stranded RNA.

As noted above, in embodiments of the invention described herein, the described methods can include administering a RIG-I inhibitor, wherein the administering comprises contacting an ocular cell of the subject with the RIG-I inhibitor. Also, as noted above, in embodiments of the invention described herein, the method can include administering a RIG-I inhibitor, wherein the administering is effective to: reduce expression of an interferon stimulated gene (ISG) in an ocular cell of the subject; reduce RIG-I protein level in an ocular cell of the subject; reduce RIG-I mRNA level in an ocular cell of the subject; reduce an IFN response in an ocular cell of the subject; reduce expression and/or secretion of a type I interferon in an ocular cell of the subject; and/or inhibit nucleic acid-induced inflammation in an ocular cell of the subject. In some embodiments, the ocular cell is a retinal ganglion (RGC) cell, a cell in the inner nuclear layer (INL), a cell in the outer nuclear layer (ONL), a retinal pigmented epithelium (RPE) cell, a cell in the choroidal layer, or a combination thereof. In particular embodiments, the ocular cell is an RPE cell. In some embodiments, the cell in the INL is a horizontal cell, a bipolar cell, an amacrine cell, an interplexiform neuron, or a Müller cell. In some embodiments, the cell in the ONL is a cone cell, a rod cell, or a photoreceptor cell.

Additionally, in some embodiments of the invention, a method described herein includes administering a RIG-I inhibitor or a pharmaceutical composition thereof to a subject, wherein the administering is effective to inhibit RPE degeneration (for example, RPE degeneration in an eye of the subject). In some embodiments of a method described herein, administering a RIG-I inhibitor is effective to inhibit transepithelial resistance impairment of an RPE (for example, an RPE of an eye of the subject).

In any of the foregoing embodiments of the invention, the subject can be a human. In some embodiments, the subject is a non-human primate, a mammal, a domesticated farm animal (for example, a horse, a cow, a pig, a goat, a sheep), a rodent (for example, a mouse or a rat), a cat, or a dog.

In some embodiments of the invention, a method described herein can be performed in vitro (for example, in a cell culture or a tissue culture). For example, in some embodiments, a method described herein can include contacting an ocular cell with a RIG-I inhibitor, wherein the cell is a cell of a cell culture or a tissue culture. In some embodiments, a method described herein (for example, a method that includes administering a RIG-I inhibitor to a subject) can be performed in vivo.

In yet another aspect of the invention, described herein are methods for modulating IFN expression or an IFN response of a cell (for example, a cell in a cell culture or a cell in a subject in need of treatment). Methods of the invention can include a step of contacting said cell with a RIG-I inhibitor. Thus, in some embodiments, disclosed herein is a method for inhibiting an IFN response in an ocular cell, the method comprising contacting the cell with a RIG-I inhibitor. In some embodiments, disclosed herein is a method for inhibiting expression of a type I IFN in an ocular cell, the method comprising contacting the cell with a RIG-I inhibitor. In some embodiments, the type I interferon is IFNβ.

In some embodiments of the invention, described herein is a method for inhibiting nucleic acid-induced inflammation (for example, RNA-induced inflammation) in an ocular cell, the method comprising contacting the cell with a RIG-I inhibitor. In some embodiments of the invention, described herein is a method for inhibiting RIG-I expression in a cell, the method comprising contacting the cell with a RIG-I inhibitor.

As noted above, embodiments of the invention described herein include a method of inhibiting an IFN response in an ocular cell, a method for inhibiting expression of a type I IFN in an ocular cell, a method for inhibiting nucleic acid-induced inflammation in an ocular cell, and a method for inhibiting RIG-I expression in a cell, wherein the method includes the step of contacting the cell (e.g., the retinal cell) with a RIG-I inhibitor. In the aforementioned embodiments of the invention, the ocular cell can be a retinal ganglion (RGC) cell, a cell in the inner nuclear layer (INL), a cell in the outer nuclear layer (ONL), a retinal pigmented epithelium (RPE) cell, or a cell in the choroidal layer. In some embodiments of the invention, the ocular cell is a retinal ganglion (RGC) cell, a cell in the INL, a cell in the ONL, a RPE cell, or a cell in the choroidal layer. In some embodiments, the ocular cell is an RPE cell or a cell in the choroidal layer. In some embodiments, the ocular cell is an RPE cell. In some embodiments, the cell in the INL is a horizontal cell, a bipolar cell, an amacrine cell, an interplexiform neuron, or a Müller cell. In some embodiments, the cell in the ONL is a cone cell, a rod cell, or a photoreceptor cell.

In certain embodiments described herein is method of inhibiting an IFN response in an ocular cell, a method for inhibiting expression of a type I IFN in an ocular cell, a method for inhibiting nucleic acid-induced inflammation in an ocular cell, and a method for inhibiting RIG-I expression in a cell, wherein the method includes the step of contacting the cell (e.g., the retinal cell) with a pharmaceutical composition comprising a RIG-I inhibitor and a pharmaceutically acceptable excipient. In the aforementioned embodiments of the invention, the ocular cell can be a retinal ganglion (RGC) cell, a cell in the inner nuclear layer (INL), a cell in the outer nuclear layer (ONL), a retinal pigmented epithelium (RPE) cell, or a cell in the choroidal layer. In some embodiments of the invention, the ocular cell is a retinal ganglion (RGC) cell, a cell in the INL, a cell in the ONL, a RPE cell, or a cell in the choroidal layer. In some embodiments, the ocular cell is an RPE cell or a cell in the choroidal layer. In some embodiments, the ocular cell is an RPE cell. In some embodiments, the cell in the INL is a horizontal cell, a bipolar cell, an amacrine cell, an interplexiform neuron, or a Müller cell. In some embodiments, the cell in the ONL is a cone cell, a rod cell, or a photoreceptor cell.

As noted above, in methods described herein, the RIG-I inhibitor can be a small molecule chemical compound, an antibody (for example, a RIG-I specific antibody), an antigen-binding fragment of a RIG-I specific antibody, a nucleic acid molecule, a peptide, or a derivative thereof. Thus, embodiments of the invention described herein include a method of inhibiting an IFN response in an ocular cell, a method for inhibiting expression of a type I IFN in an ocular cell, a method for inhibiting nucleic acid-induced inflammation in an ocular cell, and a method for inhibiting RIG-I expression in a cell, wherein the method includes the step of contacting the cell (e.g., the retinal cell) with a RIG-I inhibitor or a pharmaceutical composition comprising a RIG-I inhibitor and a pharmaceutically acceptable excipient, wherein the RIG-I inhibitor is, for example, a small molecule chemical compound, an antibody (for example, a RIG-I specific antibody), an antigen-binding fragment of a RIG-I specific antibody, a nucleic acid molecule, a peptide, or a derivative thereof. In some embodiments, the RIG-I inhibitor is a siRNA, a miRNA, a shRNA, a ribozyme, a morpholino, an antisense RNA, a triple helix forming molecule, or a sgRNA. In some embodiments of the invention described herein, the RIG-I inhibitor comprises a CRISPR system, for example, but not limited to, a RIG-I CRISPRi system or a RIG-I CRISPR-Cas9 system. In some embodiments, the RIG-I CRISPR system comprises a sgRNA. In particular embodiments, the RIG-I CRISPR-Cas9 system comprises an sgRNA comprising the sequence 5′-ACTCACCCTCCCTAAACCAG-3′ (SEQ ID NO:1), or a pharmaceutically acceptable salt thereof, or a derivative thereof. In some embodiments of the invention described herein, the RIG-I inhibitor comprises a RIG-I siRNA, for example, a RIG-I siRNA comprising a nucleotide sequence selected from the group consisting of: 5′-AAGGGAACGATTCCATCACTA-3′ (SEQ ID NO:2), 5′-TTCTACAGATTTGCTCTACTA-3′ (SEQ ID NO:3), 5′-CTCCTCCTACCCGGCTTTAAA-3′ (SEQ ID NO:4), and 5′-CAGAATTATCCCAACCGATAT-3′ (SEQ ID NO:5), or a pharmaceutically acceptable salt thereof. In some embodiments of the invention, the RIG-I inhibitor comprises a peptide or a nucleic acid molecule encoding the peptide. For example, in some embodiments of the invention, the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof. In particular embodiments described herein, the RIG-I inhibitor comprises the peptide sequence GNRDTLWHLFNTLQRRPGWVEYFI (SEQ ID NO:6), or a derivative thereof; or a nucleic acid molecule encoding the peptide of SEQ ID NO:6, or a derivative thereof. In some embodiments of the invention, the RIG-I inhibitor further comprises a vector, for example, a viral vector, for example, a lentiviral vector, an adenoviral vector, an AAV vector, a retroviral vector, or a derivative thereof.

Embodiments of the invention described herein also include a method of inhibiting an IFN response in an ocular cell, a method for inhibiting expression of a type I IFN in an ocular cell, a method for inhibiting nucleic acid-induced inflammation in an ocular cell, and a method for inhibiting RIG-I expression in a cell, wherein the method includes the step of contacting the cell (e.g., the retinal cell) with a RIG-I inhibitor or a pharmaceutical composition comprising a RIG-I inhibitor and a pharmaceutically acceptable excipient, wherein the RIG-I inhibitor comprises a small molecule compound. In some embodiments, the RIG-I inhibitor is a small molecule compound selected from the group consisting of:

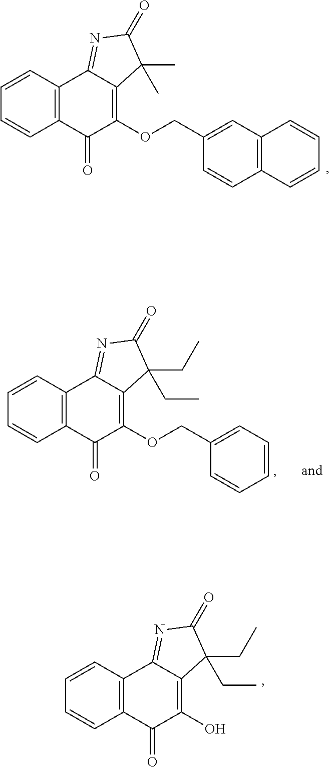 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]

or a pharmaceutically acceptable salt thereof.

In some embodiments of the invention described herein, contacting an ocular cell (e.g., a retinal cell) with a RIG-I inhibitor or a pharmaceutical composition comprising a RIG-I inhibitor and a pharmaceutically acceptable excipient produces a specific biological outcome. For example, in some embodiments, contacting an ocular cell with a RIG-I inhibitor or a pharmaceutical composition thereof reduces ocular cellular expression of a specific gene. For example, in some embodiments the contacting is effective to reduce gene expression in the cell. Thus, in some embodiments, the contacting is effective to reduce expression of an interferon stimulated gene (ISG) in the cell. In some embodiments, the contacting is effective to reduce expression of ISG15. In some embodiments, the contacting is effective to reduce a RIG-I protein level in the cell. In some embodiments, the contacting is effective to reduce a RIG-I mRNA level in the cell.

In some embodiments of the invention described herein, contacting an ocular cell (e.g., a retinal cell) with a RIG-I inhibitor or a pharmaceutical composition comprising a RIG-I inhibitor and a pharmaceutically acceptable excipient inhibits a particular cellular response. For example, in some embodiments, the contacting is effective to reduce IFN response signaling in an ocular cell. In some embodiments, the IFN response signaling comprises expression of a type I IFN. Thus, in some embodiments, the contacting is effective to reduce expression of a type I interferon in an ocular cell. In some embodiments, the contacting is effective to reduce secretion of a type I interferon in an ocular cell. In some embodiments, the type I interferon is IFNβ.

In some embodiments of the invention, contacting an ocular cell (e.g., a retinal cell) with a RIG-I inhibitor or a pharmaceutical composition comprising a RIG-I inhibitor and a pharmaceutically acceptable excipient is effective to inhibit nucleic acid-induced inflammation or inflammatory signaling in an ocular cell. For example, in some embodiments of the invention, the contacting is effective to inhibit RNA-induced inflammation or inflammatory signaling in an ocular cell of the subject.

In yet another aspect of the invention, described herein is a method for reducing toxicity of an ocular gene therapy in a subject, the method comprising administering to the subject a therapeutically effective amount of a RIG-I inhibitor or a pharmaceutical composition comprising a RIG-I inhibitor and a pharmaceutically acceptable excipient. In some embodiments, the RIG-I inhibitor comprises a small molecule chemical compound, an antibody, an antigen-binding fragment of a RIG-I antibody, a nucleic acid molecule, a peptide, or a derivative thereof. In some embodiments, the RIG-I inhibitor comprises a siRNA, a miRNA, a shRNA, a ribozyme, a morpholino, an antisense RNA, a triple helix forming molecule, or a sgRNA.

Described herein is a method for reducing toxicity of an ocular gene therapy in a subject, the method comprising administering to the subject a therapeutically effective amount of a RIG-I inhibitor or a pharmaceutical composition comprising a RIG-I inhibitor and a pharmaceutically acceptable excipient, wherein the RIG-I inhibitor comprises a CRISPR system. Thus, in some embodiments, the RIG-I inhibitor comprises a RIG-I CRISPRi system. In some embodiments, the RIG-I inhibitor comprises a RIG-I CRISPR-Cas9 system. For example, in some embodiments the RIG-I CRISPR-Cas9 system comprises an sgRNA comprising the sequence 5′-ACTCACCCTCCCTAAACCAG-3′ (SEQ ID NO: 1), or a derivative thereof.

Also described herein is a method for reducing toxicity of an ocular gene therapy in a subject, the method comprising administering to the subject a therapeutically effective amount of a RIG-I inhibitor or a pharmaceutical composition comprising a RIG-I inhibitor and a pharmaceutically acceptable excipient, wherein the RIG-I inhibitor comprises a RIG-I siRNA. In some embodiments, the RIG-I siRNA comprises a nucleotide sequence selected from the group consisting of:

(SEQ ID NO: 2)
5′ - AAGGGAACGATTCCATCACTA - 3′,
(SEQ ID NO: 3)
5′ - TTCTACAGATTTGCTCTACTA - 3′,
(SEQ ID NO: 4)
5′ - CTCCTCCTACCCGGCTTTAAA - 3′,
and
(SEQ ID NO: 5)
5′ - CAGAATTATCCCAACCGATAT - 3′.

Also described herein is a method for reducing toxicity of an ocular gene therapy in a subject, the method comprising administering to the subject a therapeutically effective amount of a RIG-I inhibitor or a pharmaceutical composition comprising a RIG-I inhibitor and a pharmaceutically acceptable excipient, wherein the RIG-I inhibitor comprises a peptide or a nucleic acid molecule encoding the peptide. For example, in some embodiments of the invention, the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof. In particular embodiments described herein, the RIG-I inhibitor comprises: the peptide sequence GNRDTLWHLFNTLQRRPGWVEYFI (SEQ ID NO:6), or a derivative thereof; or a nucleic acid molecule encoding a peptide comprising the sequence of SEQ ID NO:6, or a derivative thereof. In some embodiments of the invention, the RIG-I inhibitor further comprises a vector, for example a viral vector, for example, a lentiviral vector, an adenoviral vector, an AAV vector, a retroviral vector, or a derivative thereof.

Embodiments of the invention described herein also include a method for reducing toxicity of an ocular gene therapy in a subject, the method comprising administering to the subject a therapeutically effective amount of a RIG-I inhibitor or a pharmaceutical composition comprising a RIG-I inhibitor and a pharmaceutically acceptable excipient, wherein the RIG-I inhibitor comprises a small molecule compound. In some embodiments, the RIG-I inhibitor is a small molecule compound selected from the group consisting of:

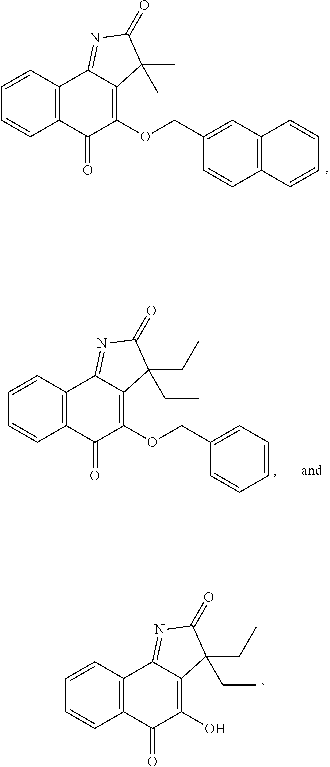 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]

or a pharmaceutically acceptable salt thereof.

In a method for reducing toxicity of an ocular gene therapy in a subject described herein, administering a RIG-I inhibitor or a pharmaceutical composition comprising a RIG-I inhibitor to a subject can be by any route considered acceptable to those of skill in the art. For example, in embodiments described herein, administration of the RIG-I inhibitor can be, but is not limited to, oral, transdermal, topical, parenteral, injection, or inhalation routes. In some embodiments of the invention, the administering is locally to an eye of a subject. Thus, in some embodiments, the administering is subconjunctival, intravitreal, periocular, retrobulbar, or intracameral. In some embodiments of the invention, the administering comprises contacting an ocular cell of the subject with the RIG-I inhibitor. For example, in some embodiments, the administering comprises contacting a retinal cell of the subject, for example, a retinal pigment epithelial cell, with the RIG-I inhibitor.

In some embodiments of the invention described herein, administering a RIG-I inhibitor or a pharmaceutical composition comprising a RIG-I inhibitor to a subject is effective to reduce expression of an interferon stimulated gene (ISG) in an ocular cell of the subject. In some embodiments, the ISG is ISG15. In some embodiments of the invention described herein, the administering is effective to reduce RIG-I protein level in an ocular cell of the subject. In some embodiments of the invention described herein, the administering is effective to reduce RIG-I mRNA level in an ocular cell of the subject. In some embodiments of the invention described herein, the administering is effective to reduce an IFN response in an ocular cell of the subject. In some embodiments, the IFN response is a type I IFN response. In some embodiments of the invention described herein, the administering is effective to reduce expression and/or secretion of a type I interferon in an ocular cell of the subject. For example, in some embodiments, the type I interferon is IFNβ. In some embodiments, the administering is effective to inhibit nucleic acid-induced inflammation in an ocular cell of the subject.

As noted above, in a method for reducing toxicity of an ocular gene therapy in a subject described herein, administering a RIG-I inhibitor or a pharmaceutical composition comprising a RIG-I inhibitor to a subject can be effective to: reduce expression of an interferon stimulated gene (ISG) in an ocular cell of the subject, reduce RIG-I protein level in an ocular cell of the subject, reduce RIG-I mRNA level in an ocular cell of the subject, reduce an IFN response in an ocular cell of the subject, reduce expression of a type I interferon in an ocular cell of the subject, reduce secretion of a type I interferon in an ocular cell of the subject, or inhibit nucleic acid-induced inflammation in an ocular cell of the subject. In some embodiments, the ocular cell is a retinal ganglion (RGC) cell, a cell in the INL, a cell in the ONL, a RPE cell, a cell in the choroidal layer, or a combination thereof. In some embodiments, the ocular cell is an RPE cell or a cell in the choroidal layer. In some embodiments, the ocular cell is an RPE cell. In some embodiments, the cell in the INL is a horizontal cell, a bipolar cell, an amacrine cell, an interplexiform neuron, or a Müller cell. In some embodiments, the cell in the ONL is a cone cell, a rod cell, or a photoreceptor cell.

In a method for reducing toxicity of an ocular gene therapy in a subject described herein, said administering a RIG-I inhibitor or a pharmaceutical composition comprising a RIG-I inhibitor can be to a human subject.

Additionally, in a method for reducing toxicity of an ocular gene therapy in a subject described herein, the gene therapy can comprise administering an AAV gene therapy vector to the subject. For example, in some embodiments administering the gene therapy comprises subretinally injecting an AAV gene therapy vector. In some embodiments, said administering the RIG-I inhibitor is effective to treat macular degeneration (AMD) associated with the gene therapy toxicity. In some embodiments, administering the RIG-I inhibitor is effective to treat geographic atrophy (GA) associated with the gene therapy toxicity. In some embodiments, administering the RIG-I inhibitor is effective to treat DME. In some embodiments, administering the RIG-I inhibitor is effective to treat abnormal angiogenesis, choroidal neovascularization (CNV), retinal vascular permeability, retinal edema, diabetic retinopathy (for example, proliferative diabetic retinopathy), neovascular (exudative) age-related macular degeneration (AMD), including CNV associated with nAMD (neovascular AMD), sequela associated with retinal ischemia, Central Retinal Vein Occlusion (CRVO), or posterior segment neovascularization.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are images showing that IFN response was elevated in GA patients. FIG. 1A shows Interferon Regulatory Transcription Factor 3 (IRF3) protein expression in non-AMD (left) control and GA (right) patients was observed using immunohistochemistry (IHC). IRF3 (red) was mainly expressed in RPE and choroid regions. FIG. 1B shows elevation of Interferon-Stimulated Gene 15 (ISG15) messenger ribonucleic acid (mRNA) levels in non-AMD control (left) and GA (right) patients were observed by RNAScope. ISG15 ribonucleic acid (RNA) signal (red dots) was low or undetected in non-AMD control patients, but significantly elevated in retinal ganglion cell (RGC), outer nuclear layer (ONL), inner nuclear layer (INL) and RPE layers of GA patients. Scale bars are shown in the figures. n=4-5 patients/group.

FIGS. 2A to 2E are graphs showing RPE cells produced and responded to IFN, particularly IFNβ. FIGS. 2A to 2C are graphs showing IFN (IFNα1, IFNβ, IFNγ, and IFNλ 1/3) production in response to nucleic acid challenge in RPE cells. Total nucleic acids were isolated from ARPE-19 cells, and cells were transfected with total nucleic acids (FIG. 2A, THP1 cells, ARPE-19 cells, or iPS-RPE cells, 0.25 μg/ml; FIG. 2B, ARPE-19 cells, 0 ng/ml-100 ng/ml; and 2C, iPS-RPE cells, 0 ng/ml-100 ng/ml). IFNβ and IFNλ were predominantly produced in RPE cells. FIGS. 2D to 2F are graphs showing ISG15 induction in RPE cells in response to stimulation with IFNα1, IFNβ, IFNγ, or IFNλ 1/2/3. Vehicle or IFNs (FIG. 2D, THP1 cells, ARPE-19 cells, or iPS-RPE cells, 100 μg/ml each; FIG. 2E, ARPE-19 cells, 0 μg/ml-100 μg/ml; and 2F, iPS-RPE cells, 0 μg/ml-100 μg/ml) were added to cells, and cells primarily responded to IFNα and IFNβ. * p<0.05 compared to relative vehicle control groups.

FIG. 3A is a series of bar graphs showing ISG15 induction 24 hours after stimulation of cells (THP-1, THP1 Cyclic GMP-AMP Synthase (cGAS) knockout (KO), ARPE-19, or induced pluripotent stem (iPS)-RPE cells) with indicated inducers (vehicle, E. coli double-stranded deoxyribonucleic acid (dsDNA-EC), G3-YSD, Poly(dA:dT), 5′ triphosphate hairpin RNA (3p-hpRNA), or Poly(I:C)) at 0.25 μg/ml. FIG. 3B is a heat map showing IFNβ release in ARPE-19 cells in which key nodes for the activation of the IFN pathway were knocked down by small interfering RNAs (siRNAs; see horizontal axis) and stimulated with indicated nucleic acids (see vertical axis; 0.25 μg/ml each). IFNβ release was measured 24 hours after nucleic acid stimulation. ARPE-19 cells were cultured 10 days after confluence for screening purposes.

FIGS. 4A and 4B are a series of bar graphs showing IFNβ (FIG. 4A) or IL6 (FIG. 4B) release measured 24 h after stimulation of cells (ARPE19-Cas9, Control guide RNA (gRNA), or RIG-I gRNA) with indicated inducers (vehicle, dsDNA-EC, G3-YSD, Poly(dA:dT), Poly(I:C), 3p-hpRNA, or ARPE19 nucleic acid (ARPE19-NA)) at 0.25 μg/ml. * p<0.05 compared to corresponded treatment groups.

FIG. 5A is a series of retinal images showing expression of RIG-I mRNA (red dots) in non-AMD control (left) and GA (right) patients observed using RNAScope. FIG. 5B is a series of images showing expression of RIG-I protein (red) in non-AMD control (left) and GA (right) patients measured using IHC. Scale bars are shown in the figures. n=4-5 patients/group.

FIG. 6A is a series of western blots showing detection of cGAS, ISG15, and p actin protein in RPE cells (THP1, THP1 cGAS KO, ARPE19, or iPS-RPE) in both basal and stimulated (0.25 μg/ml DNA or RNA) conditions. FIG. 6B is a bar graph showing detection of cGAS by ELISA in RPE cells (THP1, THP1 cGAS KO, ARPE19, or iPS-RPE) in both basal and stimulated (0.25 μg/ml DNA or RNA) conditions. cGAS antibodies were validated using THP-1 and THP-1-cGAS KO cells. FIG. 6C is a series of western blots showing detection of cGAS and Histone H3 in nuclei of indicated cell types (THP1, THP1 cGAS KO, ARPE19, or iPS-RPE) treated with sodium chloride (0, 0.5, 1, or 2 M NaCl) to dissociate proteins from chromosomes. Histone H3 was used as positive control. * p<0.05 compared to vehicle control groups. #p<0.05 compared to corresponding treatment groups between WT and cGAS KO THP1.

FIGS. 7A to 7C are graphs showing transepithelial resistance (TER, y-axis) as a function of time after stimulation (x-axis) under different conditions as measured in iPS-RPE cells cultured on transmembrane in 24-well plates. Treatment with IFNβ (FIG. 7A) or transfection with RNA but not DNA (FIG. 7B) reduced TER, and anti-IFNβ recovered IFNβ-induced (FIG. 7A) and RNA-induced barrier functional loss (FIG. 7C). FIG. 7D is a bar graph showing normalized TER after 24 h treatment with the conditions shown in FIG. 7A to 7C. * p<0.05.

FIG. 8 is a series of graphs showing expression of individual ISGs analyzed by RNAseq results of retina from AMD grade 1 and 4 patients. *p<0.05; NS, no significant difference.

FIGS. 9A to 9D provide results from KO cell line and antibody validation used in IHC and RNAScope assays. Validation for IRF3 (FIGS. 9A to 9B) or RIG-I (FIGS. 9C to 9D) antibodies for Western blot or IHC using THP1-Dual/THP1-Dual KO IRF3 cells (FIGS. 9A to 9B) and A549-Dual/A549-Dual KO RIG-I cells (FIGS. 9C to 9D). FIG. 9E shows an image of an immunoglobulin G (IgG) negative control for IHC. FIG. 9F shows a retinal image stained with a probe targeting bacterial gene DapB applied as negative control for RNAScope. No signal was observed with IgG/IHC and DapB/RNAscope.

FIG. 10A is a graph showing mRNA knockdown efficiency by various siRNAs as validated by qPCR. About ˜70% inhibition was observed in most of tested siRNAs. FIG. 10B is a schematic of Cas9 viral vector pNGx_LV_c010. FIG. 10C is an anti-flag western blot showing expression of flag-tagged Cas9 protein in transduced ARPE-19 cells. FIG. 10D is an anti-RIG-I/DDX58 western blot showing lack of detectable DDX58 protein in ARPE-19-Cas9 cells transduced with gRNA targeting DDX58/RIG-I.

### DETAILED DESCRIPTION

The present disclosure is based in part upon the identification of RIG-I (DDX58) as a key sensor for initiating a type I IFN response in RPE cells. As disclosed herein, the inventors have discovered that the RNA sensor RIG-I/DDX58 is expressed in RPE cells and senses intracellular nucleic acids (for example, ribonucleic acid (RNA)) in RPE cells. The present disclosure is also based upon the discovery that the type I IFN response functions in RPE cells in response to a nucleic acid stimulus, for example, an RNA stimulus. Thus, the inventors have surprisingly discovered that inhibitors of RIG-I are candidates for effectively reducing the IFN response. Furthermore, the inventors have discovered that inhibitors of RIG-I are promising candidates for preventing, treating, and delaying retinal degeneration. The inventors have also discovered that inhibitors of RIG-I are promising candidates for preventing, treating, and delaying AMD and GA. Additionally, the inventors have discovered that inhibitors of RIG-I are promising candidates for improving vision in subjects experiencing age-related macular degeneration (AMD) or geographic atrophy (GA).

The Examples provided herein demonstrate that the type I IFN response pathway was activated in RPE cells of GA donors. The data of the Examples demonstrate that RPE cells sense RNA, and the RNA helicase RIG-I (DDX58) was identified as a key sensor for initiating nucleic acid detection response mechanisms in RPE. The data of the Examples also demonstrate that RPE cells sense the DNA inducer poly(dA:dT), which is reported to indirectly activate IFN pathways through RIG-I via an RNA intermediary transcribed by RNA polymerase III (11, 12). The level of cGAS protein, another nucleic acid sensor, in RPE cells was below detection limits. This data suggests that RPE intrinsic nucleic acid sensing pathways are biased toward RNA sensing, while extrinsic factors from other cells may contribute to DNA sensing in retina. See, e.g. Examples 1 to 6.

The Examples also demonstrate a correlation between type I IFN response and retinal degeneration in human patient samples. RPE both produced interferon-beta (IFNβ) in response to inducers, and responded to IFNβ, leading to downstream signaling, including ISG15 induction. RPE cells showed no response to most DNA stimuli, but responded to RNA. In some instances, RPE cells showed a relatively weaker response to poly(dA:dT) as compared to RNA stimuli. In addition, RPE barrier function was perturbed by IFNβ treatment and RNA transfection. See, e.g. Examples 2 and 5.

The type I IFN system plays a critical role in host defense, especially in infectious diseases. Type I IFN responses are believed to have a role in chronic diseases with a sterile inflammation component, especially autoimmune diseases and aging-related diseases such as systemic lupus erythematosus, multiple sclerosis, Sjogren's syndrome, atherosclerosis, and Alzheimer's disease (18-22). As demonstrated in the Examples, IRF3, the master regulator for the IFN pathway, was observed in the RPE layer in human donor eye samples, indicating the machinery of IFN production is present in RPE cells. In addition, one of the core ISGs, ISG15, was significantly upregulated in GA patients, suggesting an increased type I IFN response in retina. See, e.g., Examples 1 to 3. Thus, the data provided in the Examples indicate that the type I IFN pathway is involved in pathogenesis of AMD and GA, age-related degenerative diseases.

There are many innate receptors responsible for sensing nucleotides and activating the type I IFN pathway. Toll-like receptors (TLRs), especially TLR3, are reported to recognize both extracellular and intracellular ligands to initiate an IFN response in RPE, and the TLR3 412Phe variant shows protective effects against GA (24-26). TLR3 recognizes and responds to exogenous double stranded RNAs (dsRNAs) released from damaged tissue, as well as the synthetic analog of double-stranded RNA poly(I:C), to trigger downstream type I IFN signaling (13). In the Examples provided below, however, knockdown of TLR signaling pathway members (e.g., TLR3, MYD88, and TRIF) did not affect intracellular nucleic acid sensing and IFN production, while RIG-I pathway knockdown showed inhibition, identifying RIG-I as a major sensor for intracellular RNA in RPE cells.

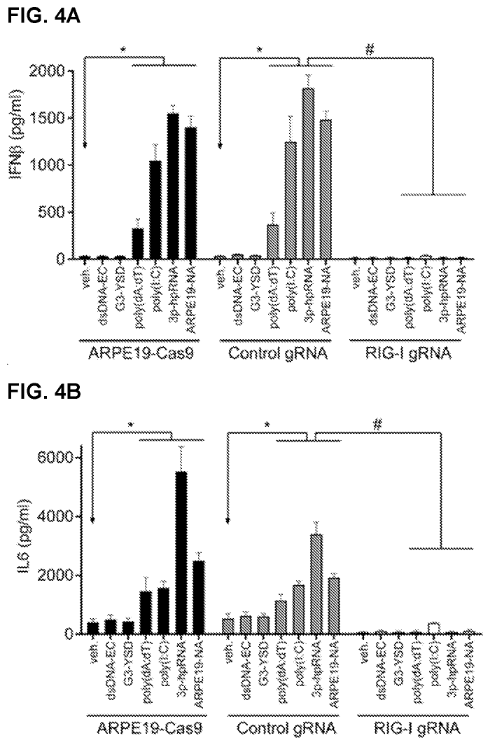

Human RIG-I, encoded by DDX58, is a dsRNA helicase enzyme that plays important roles in RNA sensing. Unexpectedly, the Examples described herein demonstrate that RIG-I, but not TLR3, is a major sensor for poly(I:C)-induced type I IFN response in RPE cells, in contrast to a previous report (13). Both TLR3 and RIG-I may be important for initiating poly(I:C)-induced, NFκB-dependent cytokine release from RPE cells (see FIG. 4B). However, as disclosed herein, RIG-I is the key sensor for initiating type I IFN response in RPE (FIG. 4A).

While type I IFN production is known to be caused by viral and bacterial infections, it may also result from chronic sterile inflammation. Endogenous nucleic acids, for example, can be abnormally released into the cytosol during cellular dysfunction and recognized by corresponding sensors. Thus, different inflammatory mediators act via specific signaling pathways (27,28). While both DNA and RNA can activate IFN pathways, the below Examples demonstrate that, in RPE cells, which are unique to the retina, RNA, but not DNA (with the exception of the RNA-intermediary acting DNA species poly(dA:dT)), induced an IFN response. There may be several potential sources of nucleic acid inducers in AMD patients. Mitochondrial dysfunction is one of the main features observed in AMD patients (29). Mitochondrial DNA (mtDNA) release from dysregulated mitochondria triggers a type I IFN response via the cGAS-STING pathway (28). Mitochondrial double-stranded RNA (mtdsRNA) can also trigger a type I IFN response through RIG-I and the RIG-I-like receptor MDA5 (27). Additionally, DICER dysregulation and Alu RNA accumulation were reported in AMD patients, and it was hypothesized that this accumulation may cause mitochondrial dysfunction, mtDNA release, and initiation of the IFN response via the cGAS-STING pathway (4).

The present disclosure is based in part, on the unexpected finding that cGAS is not involved in sensing RNA in RPE cells. As described herein, multiple methods were unable to confirm any detectable cGAS expression in RPE cells, including basal cGAS expression and following induction with RNA treatment. Thus, the results disclosed herein indicate that accumulated RNA, and not DNA, directly activated a type I IFN response in RPE cells via the RNA sensor RIG-I.

While cGAS has previously been shown to play a role in mediating nucleic acid-induced inflammatory response pathways, the inventors of the present invention have found that cGAS plays no discernible role in mediating nucleic acid-induced inflammatory responses in RPE cells. Moreover, the inventors have demonstrated that RIG-I is robustly expressed in RPE cells and that RIG-I can unexpectedly mediate a nucleic acid-induced (e.g., RNA-induced) inflammatory response in RPE cells. Not wishing to be bound by theory, it is possible that RPE cells respond to Alu RNA directly via RIG-I, which signals to professional inflammatory cells, e.g., macrophages and/or microglia, to amplify this response in a cGAS-dependent manner. It is also possible that macrophages and microglia initiate retinal damage via the cGAS/STING pathway, and then activate or prime RPE cells to be more sensitive to RNA inducers recognized by RIG-I, leading to RPE cell activation and damage. However, the Examples described herein suggest that RIG-I directly senses nucleic acid accumulation in RPE cells and induces an inflammatory response (e.g., a type I IFN response).

### Definitions

Unless defined otherwise, technical and scientific terms used herein have the same meaning as commonly understood by one of ordinary skill in the art to which the disclosure belongs. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed disclosure. In the case of conflict, the present specification, including definitions, will control. In addition, the materials, methods and examples are illustrative only and are not intended to be limiting.

As used herein, the singular forms “a”, “an” and “the” include plural references unless the content clearly dictates otherwise. Thus, for example, reference to a composition containing “a compound” can include a mixture of two or more compounds. It should also be noted that the term “or” is generally employed in its sense including “and/or” unless the content clearly dictates otherwise.

The terms “patient” and “subject” are used interchangeably herein and can refer to a mammal, including, without limitation, a human, a non-human primate (for example, a chimpanzee), a veterinary animal (e.g., cats, dogs, cows, horses, sheep, pigs, etc.) or an experimental animal model (e.g., mouse, rabbit, rat). In some embodiments, the subject is a human.

The terms “treat,” “treating”, and “treatment” of a disease, state, disorder, or condition (collectively, “disease”) include lessening, reducing, modulating, preventing, or eliminating, that results in the improvement of the condition, disease, disorder, etc. For example, treating can include: (1) preventing, delaying, or reducing the incidence and/or likelihood of the disease state or at least one clinical or sub-clinical symptom in a subject that may be afflicted with, predisposed to, or at risk for the disease, whether or not the subject is diagnosed or exhibits any such symptoms; (2) relieving the disease state, e.g., causing a temporary or permanent regression of the disease state or its clinical symptoms; (3) reducing or lessening the symptoms of the disease state; or (4) inhibiting or causing a regression of the disease or disease state, e.g., arresting, reducing or delaying the development or onset of the disease or condition, or at least one clinical or sub-clinical symptom thereof, or a relapse of the disease or any such symptom, in whole or in part. The benefit of treating or treatment to a treated subject is either statistically significant or at least observable, optionally by a quantitative method. In some embodiments described herein, a method of treating comprises administering a RIG-I inhibitor, or a pharmaceutical composition thereof, to a patient suffering from a retinal degenerative disease or a condition (for example, AMD (for example, early stage AMD, late-stage AMD, GA, wet AMD, or dry AMD), DME, abnormal angiogenesis, CNV, retinal vascular permeability, retinal edema, diabetic retinopathy (for example, proliferative diabetic retinopathy), neovascular (exudative) AMD, including CNV associated with nAMD (neovascular AMD), sequela associated with retinal ischemia, Central Retinal Vein Occlusion (CRVO), or posterior segment neovascularization).

As used herein, “preventing” or “prevent” describe reducing or eliminating the onset of the symptoms or complications of the disease, condition or disorder. The term “preventing,” when used in relation to a condition, such as AMD, is art-recognized, and refers to a method, compound, composition, or device which reduces the frequency of, or delays the onset of, signs and/or symptoms of a medical condition in a subject, for example, relative to a subject which does not receive the composition.

By “reduce” or other forms of the word, such as “reducing” or “reduction,” is meant lowering of an event or characteristic (e.g., vascular leakage). It is understood that this is typically in relation to some standard or expected value, in other words it is relative, but that it is not always necessary for the standard or relative value to be referred to.

Insofar as the methods of the present disclosure are directed to preventing disorders, it is understood that the term “prevent” does not require that the disease state be completely thwarted. Rather, as used herein, the term preventing refers to the ability of the skilled artisan to identify a patient or a population that is susceptible to disorders, such that administration of the compounds of the present disclosure may occur prior to onset of a disease. The term does not imply that the disease state be completely avoided.

The term “ameliorating a symptom” or other forms of the word such as “ameliorate a symptom” is used herein to mean that administration of a therapeutic agent of the present disclosure mitigates one or more symptoms of a disease or a disorder in a patient and/or reduces, inhibits, or eliminates a particular symptom associated with the disease or disorder prior to and/or post-administration of the therapeutic agent.

In some embodiments the disease or condition is a retinal degenerative disease or condition, for example, but not limited to, AMD or GA. In some embodiments, treatment results in reduced inflammation, reduced interferon response, reduced progression of a GA lesion, reduced size of a GA lesion, less fluid in an eye, features of ocular imaging showing improvement in eye pathology, improvement in eye pathology, improved vision, or various combination thereof. In some embodiments, a method of treating described herein (for example, a method of treating a patient in need thereof) is effective to reduce inflammation (for example, RNA-induced inflammation, for example, RNA-induced ocular (including, for example, retinal) inflammation), reduce interferon (for example, a type I IFN, for example, IFNβ) response, reduce progression of a GA lesion, reduce size of a GA lesion, reduce the amount of fluid (e.g., excess fluid) in an eye, improve eye disease (for example, AMD or GA) pathology, improvement features of ocular imaging in eye disease (for example, AMD or GA) pathology, improve vision, inhibit RPE degeneration, inhibit RPE transepithelial resistance impairment, inhibit RIG-I expression or activity, inhibit type I IFN (for example, IFNβ) secretion, inhibit type I IFN (for example, IFNβ) expression, inhibit a type I IFN (for example, IFNβ) response, or various combination thereof.

The term “in need of treatment” as used herein refers to a subject that requires or will benefit from treatment with a compound or composition described herein or a method described herein. In some embodiments, a subject in need of treatment refers to a judgment made by a physician or other caregiver that a subject requires or will benefit from treatment. This judgment can be made based on a variety of factors that are in the realm of the physician's or caregiver's expertise.

The terms “nucleic acid”, “nucleotide”, “oligonucleotide”, and “polynucleotide” encompass both DNA and RNA species unless specified otherwise.

The terms “therapeutically effective amount”, “effective amount”, and “amount effective” are used interchangeably herein to refer to an amount of a biologically or pharmaceutically active agent, either alone or as part of a pharmaceutical composition, that when administered to a subject either in a single dose or as part of a series of doses, has a detectable, positive effect on a symptom, aspect, or characteristic of a disease, disease state, disorder or condition in the subject. For example, an effective amount may be provided by one or more unit doses of an active agent described herein in any suitable formulation or dosage form, by any suitable route of administration. The therapeutically effective amount can be ascertained by measuring relevant physiological effects, and it can be adjusted in connection with the dosing regimen and diagnostic analysis of a subject's condition.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

As used herein, “pharmaceutically acceptable carrier” means buffers, carriers, and excipients suitable for use in contact with the tissues (e.g., ocular, for example, retinal tissue) of human beings and animals without excessive toxicity, irritation, allergic response, or other problem or complication, commensurate with a reasonable benefit/risk ratio. The carrier(s) should be “acceptable” in the sense of being compatible with the other ingredients of the formulation and not deleterious to the recipient. Pharmaceutically acceptable carriers include buffers, solvents, dispersion media, coatings, isotonic and absorption-delaying agents, and the like, that are compatible with pharmaceutical administration. The use of such media and agents for pharmaceutically active substances is known in the art. In certain embodiments the pharmaceutical composition is administered ocularly, topically, by injection, locally, systemically, subconjunctivally, intravitreally, periocularly, retrobulbarly, intracamerally, or parenterally.

The term “about” means within an acceptable error range for the particular variable or value as determined by one of ordinary skill in the art, which will depend in part on how the variable or value is measured or determined, e.g., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given variable or value, again per the practice of the relevant art.

If aspects or embodiments of the disclosure are described as “comprising”, or versions there of (e.g., comprises), a feature, embodiments also are contemplated as “consisting of” or “consisting essentially of” the feature.

As used herein, the term “derivative” or “derived from” in the context of proteins, oligonucleotides, polypeptides, or nucleic acid molecules refer to: (a) a polypeptide or nucleic acid molecule that has at least 80%, 85%, 90%, 91%, 92%, 93%, 94%, 95%, 96%, 97%, 98% or 99% sequence identity to the polypeptide or nucleic acid molecule it is a derivative of; (b) a polypeptide encoded by a nucleotide sequence that has at least 80%, 85%, 90%, 91%, 92%, 93%, 94%, 95%, 96%, 97%, 98% or 99% sequence identity to a nucleotide sequence encoding the polypeptide it is a derivative of; (c) a polypeptide or nucleic acid molecule that contains 1, 2, 3, 4, 5, 6, 7, 8, 9, 10, 11, 12, 13, 14, 15, 16, 17, 18, 19, or 20 mutations (e.g., additions, deletions and/or substitutions) relative to the polypeptide or nucleic acid molecule it is a derivative of; or (d) a fragment of the polypeptide or nucleic acid molecule that comprises about or at least about 10, 20, 30, 40, 50, 60, 70, 80, 90, 100, 150, 200, 250, 300, 350, 400, 450, 500, or 1000 contiguous amino acids or nucleotides from the polypeptide or nucleic acid molecule.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector,” “recombinant vector,” and “expression vector” as used herein refer to the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, an adenoviral, adeno-associated, alphaviral, herpes, lentiviral, retroviral, or vaccinia vector. In some embodiments, the vector is a lentiviral vector.

As used herein, the term “inhibitor” refers to a substance that interferes with (e.g., inhibits, reduces, eliminates, or suppresses) a target molecule or target compound or target process, such as an inflammatory response (for example, suppresses an active signaling pathway promoting inflammatory protein secretion, thereby inducing inflammation) when compared to an untreated cell or a cell treated with a compound that does not inhibit a treated cell or tissue. In some embodiments, an inhibitor is a small molecule, a protein, a fusion protein, a peptide, a polynucleotide (e.g., antisense, Crispr guide RNA, siRNA, microRNA (miRNA), small hairpin RNA (shRNA), Dicer-substrate short interfering RNA (DsiRNA), long small interfering RNA (IsiRNA), single stranded siRNA (ss-siRNA), Piwi-interacting RNA (piRNA), endogenous short interfering RNA (endo-siRNA), an asymmetric siRNA (asiRNA)), a nucleotide, an aptamer, an avimer, or a derivative or fragment of any of the proceeding molecules. In some embodiments, an inhibitor described herein is an antibody or an antigen-binding antibody fragment that binds specifically to a protein or peptide, for example, an antibody or an antigen-binding antibody fragment that specifically binds RIG-I. In some embodiments, the antibody is a fully human antibody or a humanized antibody. In some embodiments, the antigen-binding antibody fragment is selected from the group of: a Fab fragment, a F(ab′)2 fragment, and a scFv fragment. In some embodiments, the RIG-I inhibitor comprises a vector that includes a nucleotide sequence encoding, for example, a protein, a fusion protein, a peptide, a polynucleotide, a nucleotide, or a derivative or fragment of any of the proceeding molecules.

As used herein, “RIG-I” (also known as DDX58, RIG1; RIGI; RLR-1; and SGMRT2) refers alternatively to the gene sequence of NCBI Gene ID No. 23586 or a nucleotide or protein sequence encoded by the gene sequence of NCBI Gene ID No. 23586, or an ortholog thereof. Nucleotide sequences encoded by RIG-I include, for example, the RIG-I mRNA sequence of NCBI Reference Sequence: NM_014314.4 (SEQ ID NO:12). Protein sequences encoded by RIG-I include, for example, the RIG-I protein sequence of NCBI Reference Sequence: NP_055129.2 (SEQ ID NO:13).

A RIG-I inhibitor can be a RIG-I antagonist. Inhibition of a target gene can result in reduced transcription of the gene and a reduced level of mRNA transcribed from the target gene in the cell. Inhibition of the target gene or target protein or peptide can also result in hindered translation of mRNA encoding the target protein or peptide. As described herein, the inhibitor can comprise a small molecule compound, a peptide, a nucleic acid molecule, or a combination thereof. In some embodiments, a RIG-I inhibitor increases the rate of RIG-I mRNA degradation or of RIG-I protein degradation in a cell. In some embodiments, the inhibitor knocks down or knocks out RIG-I gene expression. In some embodiments, the RIG-I inhibitor comprises a siRNA, a miRNA, a shRNA, a ribozyme, a morpholino, an antisense RNA, a triple helix forming molecule, or a sgRNA. In some embodiments, a RIG-I inhibitor may include an enzyme or regulatory protein, such as, but not limited to, LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof. In some embodiments, a RIG-I inhibitor is a CRISPR system (e.g., a RIG-I CRISPR-Cas9 system or a RIG-I CRISPRi system).

In some embodiments, a RIG-I inhibitor described herein can be a RIG-I inhibitor, for example, a RIG-I inhibitor peptide, disclosed in International Patent Publication No. WO 2013/016278, which is herein incorporated by reference. For example, in some embodiments, a method described herein comprises contacting a cell or administering a RIG-I inhibitor comprising the peptide sequence of SEQ ID NO:6 (GNRDTLWHLFNTLQRRPGWVEYFI), or a derivative thereof; or a nucleic acid molecule encoding the peptide of SEQ ID NO:6, or a derivative thereof. In some embodiments, a method described herein comprises contacting a cell or administering a RIG-I inhibitor comprising a polypeptide fragment of human Mitochondrial Antiviral Signaling (MAVS) protein. For example, in some embodiments, the RIG-I inhibitor is a polypeptide comprising one or more of the following groups of amino acids of human MAVS protein, identified by UniProtKB Identification No. Q7Z434 (SEQ ID NO:7): 10-77, 15-77, 20-77, 25-77, 30-77, 35-77, 40-77, 45-77, 50-77, 10-73, 15-73, 20-73, 25-73, 30-73, 35-73, 40-73, 45-73, or 50-73.

The terms and expressions employed herein are used as terms of description and not of limitation, and use of such terms and expressions do not exclude any equivalents of the features shown and described or portions thereof, and various modifications are possible within the scope of the technology claimed.

The practice of the present disclosure employs, unless otherwise indicated, conventional techniques of statistical analysis, molecular biology (including recombinant techniques), microbiology, cell biology, and biochemistry, which are within the skill of one who practices the art. Such tools and techniques are described in detail in, e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

### Methods of the Invention

In one aspect, the present invention provides a method for treating a retinal degenerative disease or condition in a subject in need thereof. The method includes administering to the subject a therapeutically effective amount of a retinoic acid-inducible gene I (RIG-I) inhibitor.

In another aspect, the present invention provides for a method for improving vision in a subject. The method includes administering to the subject a therapeutically effective amount of the RIG-I inhibitor.

In a further aspect, the present invention provides a method for reducing the progression of GA lesion enlargement in a subject. The method comprises administering to the subject a therapeutically effective amount of the RIG-I inhibitor.

In some embodiments the present invention provides for methods for administering a RIG-I inhibitor to a subject to prevent the development of a retinal degenerative disease. In some embodiments, the subject is at risk of developing the retinal degenerative disease. In some embodiments, the subject has a genetic predisposition for developing the retinal degenerative disease.

In various embodiments the retinal degenerative disease or condition is age-related macular degeneration (AMD). The AMD can be geographic atrophy (GA). Further non-limiting examples of retinal degenerative diseases include retinitis pigmentosa, diabetic macular degeneration (DME), macular dystrophies, inherited macular degeneration, inherited retinal degeneration, abnormal angiogenesis, choroidal neovascularization (CNV), retinal vascular permeability, retinal edema, diabetic retinopathy (for example, proliferative diabetic retinopathy), neovascular (exudative) age-related macular degeneration (AMD), including CNV associated with nAMD (neovascular AMD), sequela associated with retinal ischemia, Central Retinal Vein Occlusion (CRVO), and posterior segment neovascularization.

In some embodiments, the subject has impaired vision resulting from a retinal tear, retinal detachment, diabetic retinopathy, or epiretinal membrane. In some embodiments the subject has impaired vision resulting from an ocular autoimmune disease or chronic inflammation, optionally chronic sterile inflammation.

In various embodiments, the RIG-I inhibitor is administered in an amount effective to reduce an IFN response in an ocular cell of the subject. The ocular cell can be a retinal ganglion (RGC) cell, a cell in the inner nuclear layer (INL), a cell in the outer nuclear layer (ONL), a retinal pigmented epithelium (RPE) cell, a cell in the choroidal layer, or a combination thereof. In some embodiments, the ocular cell is an RPE cell or a choroidal cell. In some embodiments, the ocular cell is an RPE cell. In some embodiments the cell in the INL is a horizontal cell, a bipolar cell, an amacrine cell, an interplexiform neuron, or a Müller cell. In some embodiments, the cell in the ONL is a cone cell, a rod cell, or a photoreceptor cell.

The IFN response can be a type I IFN response. Type I IFN response may be measured as expression or enhanced expression of interferon-stimulated genes (ISGs), presence or elevated levels of peptides encoded by ISGs, expression or elevated expression of RIG-I, presence or elevated levels of RIG-I protein, or various combinations thereof. Expression of a gene can be measured by quantifying or detecting levels of mRNA in a cell that has been transcribed from the gene, e.g. by qRT-PCR, a DNA microarray, in-situ hybridization, northern blot, RNA-Seq and various other techniques known in the art. Protein levels can be measured by any of various techniques known in the art, e.g. by immunoblotting, proteomics, or mass spectrometry. In various embodiments, a gene is considered as expressed by a cell when mRNA transcribed from the gene is at detectable levels within the cell. Enhanced or elevated expression of a gene can be defined as expression that is greater than expression measured in a healthy cell. Elevated levels of a protein can be defined as levels greater than those measured in a healthy cell. A healthy cell can be obtained from or correspond to a subject not at risk of developing and/or that has not developed an eye disease, optionally a retinal degenerative disease.

In some embodiments, the IFN response is measured as expression of an ISG or RIG-I at levels of about or of at least about 1.01, 1.02, 1.03, 1.04, 1.05, 1.06, 1.07, 1.08, 1.09, 1.1, 1.2, 1.3, 1.4, 1.5, 1.6, 1.7, 1.8, 1.9, 2, 2.1, 2.2, 2.3, 2.4, 2.5, 2.75, 3, 4, 5, 6, 7, 8, 9, or 10-fold greater than levels observed in a healthy cell. In some embodiments, the IFN response is measured as the presence of RIG-I protein or ISG protein at levels of about or of at least about 1.01, 1.02, 1.03, 1.04, 1.05, 1.06, 1.07, 1.08, 1.09, 1.1, 1.2, 1.3, 1.4, 1.5, 1.6, 1.7, 1.8, 1.9, 2, 2.1, 2.2, 2.3, 2.4, 2.5, 2.75, 3, 4, 5, 6, 7, 8, 9, or 10-fold greater than levels observed in a healthy cell.

In various embodiments, administration of the RIG-I inhibitor results in a reduction in the expression of an ISG in the ocular cell. In some embodiments, the ISG is ISG15. In some embodiments, administration of the RIG-I inhibitor results in a reduction in RIG-I protein levels in the ocular cell. In some embodiments, administration of the RIG-I inhibitor results in a reduction in RIG-I mRNA levels in the ocular cell. In some embodiments, administration of the RIG-I inhibitor results in improved vision in the subject. In some embodiments, administration of the RIG-I inhibitor results in reversal or slowing of progression of a retinal degenerative disease in the subject.

RIG-I Inhibitors

The RIG-I inhibitor can comprise a small molecule chemical compound, an antibody, an antigen-binding fragment of a RIG-I antibody, a nucleic acid molecule, a peptide, or derivatives thereof. In various embodiments, the RIG-I inhibitor knocks down and/or knocks out RIG-I. The RIG-I inhibitor can comprise molecules effective for use in an RNA interference (RNAi) technology. The RIG-I inhibitor can comprise siRNA, miRNA, shRNA, a ribozyme, a morpholino, antisense RNA/DNA, a triple helix forming molecule, or sgRNA.

Several different types of molecules can be used effectively in RNAi technology. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g., as an antiviral mechanism or in shaping the chromatin structure of a genome. Synthetic siRNAs have been shown to be able to induce RNAi in mammalian cells.

MicroRNA (miRNA) are a related class of gene regulatory small RNAs, typically 21-23 nt in length. They typically differ from siRNA because they are processed from single stranded RNA precursors and show only partially complementary to mRNA targets. Initial studies have indicated that miRNAs regulate gene expression post-transcriptionally at the level of translational inhibition at P-Bodies in the cytoplasm. However, miRNAs may also guide mRNA cleavage similar to siRNAs. This is often the case in plants where the target sites are typically highly complementary to the miRNA. While target sites in plant mRNAs can be found in the 5′UTR, open-reading frames and 3′UTR, in animals, it is the 3′ UTR that is the main target. miRNAs are first transcribed as part of a primary microRNA (pri-miRNA). This is then processed by the Drosha with the help of Pasha/DGCR8 (=Microprocessor complex) into pre-miRNAs. The ca. 75 nt pre-miRNA is then exported to the cytoplasm by exportin-5, where it is then diced into 21-23 nt siRNA-like molecules by Dicer. In some cases, multiple miRNAs can be found on the pri-miRNA.

Short hairpin RNA (shRNA) is yet another type of RNA that may be used to effect RNAi. It is a sequence of RNA that makes a tight hairpin turn that can be used to silence gene expression. shRNA is transcribed by RNA polymerase Ill.

Short-interfering RNAs (siRNAs) and short-hairpin RNAs (shRNAs) are extensively used to silence various genes to silence functions carried out by the genes. It is becoming easier to harness RNAi to silence specific genes, owing to the development of libraries of readymade shRNA and siRNA gene-silencing constructs by using a variety of sources. For example, RNAi Codex, which consists of a database of shRNA related information and an associated website, has been developed as a portal for publicly available shRNA resources. RNAi Codex currently holds data from the Hannon-Elledge shRNA library and allows the use of biologist-friendly gene names to access information on shRNA constructs that can silence the gene of interest. It is designed to hold user-contributed annotations and publications for each construct, as and when such data become available. Olson et al. (Nucleic Acids Res. 34(Database issue): D153-D157, 2006, incorporated by reference) have provided detailed descriptions about features of RNAi Codex, and have explained the use of the tool. All this information may be used to help design various siRNAs or shRNAs targeting RIG-I.

In some embodiments, the RIG-I inhibitor comprises a RIG-I siRNA. In some embodiments, the RIG-I inhibitor comprises a RIG-I shRNA. Non-limiting examples of RIG-I siRNA or RIG-I shRNA molecules suitable for use in the present invention include those described in He, T. et al. 2019. Life Sci. 131: 116570; Kang, Y G. et al. 2019. Nucleic Acid Ther. 29: 291-299; Li, L. et al. 2018. Neurosci. Lett. 672: 46-52; Raicevic, G. et al. 2017. Sci Rep. 7: 2896; Liu, Y. et al. 2016. Journal of virology; Li, P. et al. 2016. Nature medicine. 22: 807-11; Yu, L. et al. 2016. Biomed Res Int. 2016: 9872138; Chiang, C. et al. 2015. Journal of virology. 89: 8011-25; Webster Marketon, J I. et al. 2014; Virology. 449: 62-9, Harashima, N. et al. 2014; Mol. Cancer. 13: 217; and Goulet, M L. et al. 2013. PLoS pathogens. 9: e1003298, all of which are incorporated herein by reference to provide examples of RIG-I shRNA and RIG-I shRNA sequences. In embodiments described herein, the RIG-I siRNA can comprise the nucleotide sequence of

(SEQ ID NO: 2)
5′ - AAGGGAACGATTCCATCACTA - 3′,
(SEQ ID NO: 3)
5′ - TTCTACAGATTTGCTCTACTA - 3′,
(SEQ ID NO: 4)
5′ - CTCCTCCTACCCGGCTTTAAA - 3′,
and
(SEQ ID NO: 5)
5′ - CAGAATTATCCCAACCGATAT - 3′.

In some embodiments, the RIG-I inhibitor is a ribozyme. Ribozyme molecules designed to catalytically cleave a target mRNA transcript can also be used to prevent translation of RIG-I. Accordingly, in another embodiment, the compositions of the invention comprise ribozymes specifically directed to the mRNA of RIG-I. Ribozymes are enzymatic RNA molecules capable of catalyzing the specific cleavage of RNA. (For a review, see Rossi, Current Biology 4: 469-471, 1994). The mechanism of ribozyme action involves sequence specific hybridization of the ribozyme molecule to complementary target RNA, followed by an endonucleolytic cleavage event. The composition of ribozyme molecules preferably includes one or more sequences complementary to a target mRNA, and the well-known catalytic sequence responsible for mRNA cleavage or a functionally equivalent sequence (see, e.g., U.S. Pat. No. 5,093,246, incorporated herein by reference in its entirety).

Ribozymes that cleave mRNA at site specific recognition sequences can be used to destroy target mRNAs. Also, hammerhead ribozymes cleave mRNAs at locations dictated by flanking regions that form complementary base pairs with the target mRNA. In some embodiments, the target mRNA has the following sequence of two bases: 5′-UG-3′. The construction and production of hammerhead ribozymes is described more fully in Haseloff and Gerlach, Nature 334: 585-591, 1988; and see PCT Appln. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., Proc. Natl. Acad. Sci. USA, 92: 6175-79, 1995; de Feyter, and Gaudron, Methods in Molecular Biology, Vol. 74, Chapter 43, “Expressing Ribozymes in Plants,” Edited by Turner, P. C, Humana Press Inc., Totowa, N.J.). In particular, RNA polymerase III-mediated expression of tRNA fusion ribozymes are known in the art (see, Kawasaki et al., Nature 393: 284-9, 1998; Kuwabara et al., Nature Biotechnol. 16: 961-5, 1998; and Kuwabara et al., Mol. Cell. 2: 617-27, 1998; Koseki et al., J Virol 73: 1868-77, 1999; Kuwabara et al., Proc Natl Acad Sci USA 96: 1886-91, 1999; Tanabe et al., Nature 406: 473-4, 2000). There are typically a number of potential hammerhead ribozyme cleavage sites within a given target sequence. In some embodiments, the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the C-terminal amino acid domains of, for example, long and short forms of target would allow the selective targeting of one or the other form of the target, and thus, have a selective effect on one form of the target gene product.

Gene targeting ribozymes contain a hybridizing region complementary to two regions, each of at least 5 and preferably each 6, 7, 8, 9, 10, 11, 12, 13, 14, 15, 16, 17, 18, 19 or 20 contiguous nucleotides in length of a target mRNA, such as an mRNA of a sequence represented in the RIG-I gene. In addition, ribozymes possess highly specific endoribonuclease activity, which autocatalytically cleaves the target sense mRNA. The present invention extends to ribozymes which hybridize to a sense mRNA encoding RIG-I, thereby hybridizing to the sense mRNA and cleaving it, such that it is no longer capable of being translated to synthesize a functional polypeptide product.

The ribozymes used as RIG-I inhibitors in the methods of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al., Science 224:574-578, 1984; Zaug et al., Science 231: 470-475, 1986; Zaug et al., Nature 324: 429-433, 1986; published International patent application No. WO88/04300 by University Patents Inc.; Been, et al., Cell 47: 207-216, 1986). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. In some embodiments, a method of delivery of the ribozyme involves using a DNA construct “encoding” the ribozyme under the control of a strong constitutive pol Ill or pol 11 promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. The gene-targeting portions of the ribozyme or RNAi may be substantially the same sequence of at least 5 and preferably 6, 7, 8, 9, 10, 11, 12, 13, 14, 15, 16, 17, 18, 19 or 20 or more contiguous nucleotides of a target nucleic acid, such as the RIG-I gene. In a long target RNA chain, significant numbers of target sites are not accessible to the ribozyme because they are hidden within secondary or tertiary structures (Birikh et al., Eur J Biochem 245: 1-16, 1997). To overcome the problem of target RNA accessibility, computer generated predictions of secondary structure are typically used to identify targets that are most likely to be single-stranded or have an “open” configuration (see Jaeger et al., Methods Enzymol 183: 281-306, 1989). Other approaches utilize a systematic approach to predicting secondary structure which involves assessing a huge number of candidate hybridizing oligonucleotides molecules (see Milner et al., Nat Biotechnol 15: 537-41, 1997; and Patzel and Sczakiel, Nat Biotechnol 16: 64-8, 1998). Additionally, U.S. Pat. No. 6,251,588, the contents of which are hereby incorporated herein, describes methods for evaluating oligonucleotide probe sequences so as to predict the potential for hybridization to a target nucleic acid sequence. In some embodiments, a method of the invention provides for the use of such methods to select preferred segments of a target mRNA sequence that are predicted to be single-stranded and, further, for the opportunistic utilization of the same or substantially identical target mRNA sequence, optionally comprising about 10-20 consecutive nucleotides of the target mRNA, in the design of both the RNAi oligonucleotides and ribozymes of the invention.

In some embodiments, the RIG-I inhibitor comprises an antisense nucleic acid. In some embodiments, isolated “antisense” nucleic acids can be used to inhibit expression of RIG-I, e.g., by inhibiting transcription and/or translation of RIG-I nucleic acids. The antisense nucleic acids may bind by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

An antisense construct of the present invention can be delivered, for example, as a vector which, when transcribed in the cell, produces RNA which is complementary to at least a unique portion of the cellular mRNA which encodes a RIG-I polypeptide. Alternatively, the antisense construct is an oligonucleotide probe, which is generated ex vivo and which, when introduced into the cell causes inhibition of expression by hybridizing with the mRNA and/or genomic sequences of a target nucleic acid. Such oligonucleotide probes may be modified oligonucleotides, which are resistant to endogenous nucleases, e.g., exonucleases and/or endonucleases, and are therefore stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al., BioTechniques 6: 958-976, 1988; and Stein et al., Cancer Res 48: 2659-2668, 1988.

With respect to antisense DNA, oligodeoxyribonucleotides derived from the translation initiation site, e.g., between the −10 and +10 regions of the target gene, are preferred in some embodiments. Antisense approaches involve the design of oligonucleotides (either DNA or RNA) that are complementary to mRNA encoding the RIG-I protein. The antisense oligonucleotides will bind to the mRNA transcripts and prevent translation. Absolute complementarity, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, may work most efficiently at inhibiting translation. However, sequences complementary to the 3′ untranslated sequences of mRNAs have been shown to be effective at inhibiting translation of mRNAs as well (Wagner, Nature 372: 333, 1994). Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA can include the complement of the AUG start codon. Antisense oligonucleotides complementary to mRNA coding regions could also be used in accordance with the invention. Whether designed to hybridize to the 5′, 3′ or coding region of mRNA, antisense nucleic acids can be at least six nucleotides in length, optionally at least about 100 nucleotides in length and/or less than about 150, 50, 25, 17 or 10 nucleotides in length.

In some embodiments, in vitro studies are first performed to quantitate the ability of the antisense oligonucleotide to inhibit gene expression. These studies can utilize controls that distinguish between antisense gene inhibition and nonspecific biological effects of oligonucleotides. These studies can compare levels of the target RNA or protein with that of an internal control RNA or protein. Results obtained using the antisense oligonucleotide may be compared with those obtained using a control oligonucleotide. The control oligonucleotide may be of approximately the same length as the test oligonucleotide and that the nucleotide sequence of the oligonucleotide differs from the antisense sequence no more than is necessary to prevent specific hybridization to the target sequence.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., Proc. Natl. Acad. Sci. U.S.A. 86: 6553-6556, 1989; Lemaitre et al., Proc. Natl. Acad. Sci. 84: 648-652, 1987; PCT Publication No. WO88/09810) or the blood-brain barrier (see, e.g., PCT Publication No. WO89/10134), hybridization-triggered cleavage agents (see, e.g., Krol et al., BioTechniques 6: 958-976, 1988) or intercalating agents (see, e.g., Zon, Pharm. Res. 5: 539-549, 1988). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose. The antisense oligonucleotide can also contain a neutral peptide-like backbone. Such molecules are termed peptide nucleic acid (PNA)-oligomers and are described, e.g., in Perry-O'Keefe et al., Proc. Natl. Acad. Sci. U.S.A. 93: 14670, 1996, and in Eglom et al., Nature 365: 566, 1993. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an alpha-anomeric oligonucleotide. An alpha-anomeric oligonucleotide forms specific double-stranded hybrids with complementary RNA in which, contrary to the usual antiparallel orientation, the strands run parallel to each other (Gautier et al., Nucl. Acids Res. 15: 6625-6641, 1987). The oligonucleotide is a 2′-O-methylribonucleotide (Inoue et al., Nucl. Acids Res. 15: 6131-6148, 1987), or a chimeric RNA-DNA analogue (Inoue et al., FEBS Lett. 215: 327-330, 1987).

While antisense nucleotides complementary to the coding region RIG-I mRNA sequence can be used, those complementary to the transcribed untranslated region may also be used.

In some embodiments, RIG-I gene expression can be reduced by targeting deoxyribonucleotide sequences complementary to the regulatory region of the gene (i.e., the promoter and/or enhancers) to form triple helical structures that prevent transcription of the gene in target cells in the body (see generally, Helene, Anticancer Drug Des. 6(6): 569-84, 1991; Helene et al., Ann. N.Y. Acad. Sci., 660: 27-36, 1992; and Maher, Bioassays 14(12): 807-15, 1992).

In some embodiments, the RIG-I inhibitor comprises a triple helix forming molecule. Nucleic acid molecules to be used in triple helix formation for the inhibition of transcription can be single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules can form a triple helix with a DNA duplex that is rich in GC pairs, in which the majority of the purine residues are located on a single strand of the targeted duplex, resulting in CGC triplets across the three strands in the triplex.

Potential target sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

In certain embodiments, the antisense oligonucleotides are morpholino antisenses. Morpholinos are synthetic molecules which are the product of a redesign of natural nucleic acid structure. Usually 25 bases in length, they bind to complementary sequences of RNA by standard nucleic acid base-pairing. Structurally, the difference between morpholinos and DNA is that while morpholinos have standard nucleic acid bases, those bases are bound to morpholine rings instead of deoxyribose rings, and linked through phosphorodiamidate groups instead of phosphates. Replacement of anionic phosphates with the uncharged phosphorodiamidate groups eliminates ionization in the usual physiological pH range, so morpholinos in organisms or cells are uncharged molecules. Morpholinos are not chimeric oligos; the entire backbone of a morpholino is made from these modified subunits. Morpholinos are most commonly used as single-stranded oligos, though heteroduplexes of a morpholino strand and a complementary DNA strand may be used in combination with cationic cytosolic delivery reagents.

Unlike many antisense structural types (e.g., phosphorothioates), morpholinos do not degrade their target RNA molecules. Instead, morpholinos act by “steric blocking,” binding to a target sequence within an RNA and simply getting in the way of molecules which might otherwise interact with the RNA. Morpholino oligos are often used to investigate the role of a specific mRNA transcript in an embryo, such as eggs or embryos of zebrafish, African clawed frog (Xenopus), chick, and sea urchin, producing morphant embryos. With appropriate cytosolic delivery systems, morpholinos are effective in cell culture.

Bound to the 5′-untranslated region of messenger RNA (mRNA), morpholinos can interfere with progression of the ribosomal initiation complex from the 5′ cap to the start codon. This prevents translation of the coding region of the targeted transcript (called “knocking down” gene expression). Some morpholinos knock down expression so effectively that after degradation of preexisting proteins the targeted proteins become undetectable by Western blot.

Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety and acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products.

Morpholinos have also been used to block miRNA activity, ribozyme activity, intronic splice silencers, and splice enhancers. U2 and U12 snRNP functions have been inhibited by Morpholinos. Morpholinos targeted to “slippery” mRNA sequences within protein coding regions can induce translational frameshifts. Activities of Morpholinos against this variety of targets suggest that Morpholinos can be used as a general-purpose tool for blocking interactions of proteins or nucleic acids with mRNA.

In some embodiments, the present invention provides methods and compositions for gene editing/cloning utilizing DNA nucleases. CRISPR complexes, transcription activator-like effector nucleases (TALENs), zinc finger nucleases (ZFNs), and Fokl restriction enzymes are some of the sequence-specific nucleases that have been used as gene editing tools. These enzymes are able to target their nuclease activities to desired target loci through interactions with guide regions engineered to recognize sequences of interest. In some embodiments, the present disclosure teaches CRISPR-based gene editing methods.

The principles of in vivo CRISPR-based editing largely rely on natural cellular DNA repair systems. Double-stranded dsDNA breaks introduced by nucleases are repaired by either non-homologous end-joining (NHEJ) or homology-directed repair (HDR), or single strand annealing, (SSA), or microhomology end joining (MMEJ).

HDR relies on a template DNA containing sequences homologous to the region surrounding the targeted site of DNA cleavage. Cellular repair proteins use the homology between the exogenously supplied or endogenous DNA sequences and the site surrounding a DNA break to repair the dsDNA break, replacing the break with the sequence on the template DNA. Failure to integrate the template DNA, however, can result in NHEJ, MMEJ, or SSA. NHEJ, MMEJ and SSA are error-prone processes that are often accompanied by insertion or deletion of nucleotides (indels) at the target site, resulting in genetic knockout (silencing) of the targeted region of the genome due to frameshift mutations or insertions of a premature stop codon. Cpf1-mediated editing can also function via traditional hybridization of overhangs created by the endonuclease, followed by ligation.

CRISPR (Clustered Regularly Interspaced Short Palindromic Repeats) and CRISPR-associated (cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al., Nat. Struct. Mol. Biol. 2001:18; 688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in class 2 systems all functions of the effector complex are carried out by a single protein (e.g., Cas9 or Cpf1). In some embodiments, the present disclosure teaches using type II and/or type V single-subunit effector systems. Thus, in some embodiments, the present disclosure teaches using class 2 CRISPR systems.

In some embodiments, the present disclosure teaches methods of gene editing using a Type II CRISPR system. In some embodiments, the Type II CRISPR system (CRISPR-Cas9) uses the Cas9 enzyme. Type II systems rely on a i) single endonuclease protein, ii) a transactivating crRNA (tracrRNA), and iii) a crRNA where a ˜20-nucleotide (nt) portion of the 5′ end of crRNA is complementary to a target nucleic acid. The region of a CRISPR crRNA strand that is complementary to its target DNA protospacer is hereby referred to as “guide sequence” (sgRNA).

Cas9 endonucleases produce blunt end DNA breaks and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et. al., Science. 2012:337; 816-821). In some embodiments, the PAM motif recognized by a Cas9 varies for different Cas9 proteins.

In some embodiments, one skilled in the art can appreciate that the Cas9 disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27; 156(5):935-49; Jinek M. et al. Science. 2012 337:816-21; and Jinek M. et al. Science. 2014 Mar. 14; 343(6176); see also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference. Thus, in some embodiments, the systems and methods disclosed herein can be used with the wild type Cas9 protein having double-stranded nuclease activity, Cas9 mutants that act as single stranded nickases, or other mutants with modified nuclease activity.

The present disclosure further envisions the use of catalytically inactivated Cas9 mutants, as described in further detail below. In some embodiments, the term “catalytically inactivated” or “catalytically inactive” CRISPR refers to a CRISPR protein in which the DNAase catalytic domain is non-functional (i.e., the enzyme no longer cleaves DNA). Thus in some embodiments, the present disclosure teaches dCas9 mutants. A non-limiting list of mutations that reduce or eliminate nuclease in Cas9 includes: D10, G12, G17, E762, H840, N854, N863, H982, H983, A984, D986, or A987, or a mutation in a corresponding location in a Cas9 homologue or ortholog. The mutation(s) can include substitution with any natural (e.g., alanine) or non-natural amino acid, or deletion. An exemplary nuclease defective dCas9 protein is Cas9D10A&H840A (Jinek, et al., Science. 2012 Aug. 17; 337(6096):816-21; Qi, et al., Cell. 2013 Feb. 28; 152(5):1173-83).

In some embodiments, the RIG-I inhibitor comprises a RIG-I CRISPR-Cas9 system. The RIG-I CRISPR-Cas9 system can comprise an sgRNA. In some embodiments, the sgRNA comprises the sequence ACTCACCCTCCCTAAACCAG (SEQ ID NO: 1), or a derivative thereof. In some embodiments, the sgRNA comprises a sequence with about or at least about 80%, 85%, 86%, 87%, 88%, 89%, 90%, 91%, 92%, 93%, 94%, 95%, 96%, 97%, 98%, or 99% identity to SEQ ID NO: 1.

In other embodiments, the present disclosure teaches methods of gene editing using a Type V CRISPR system. In some embodiments, the present disclosure teaches methods of using CRISPR from Prevotella and Francisella 1 (Cpf1).

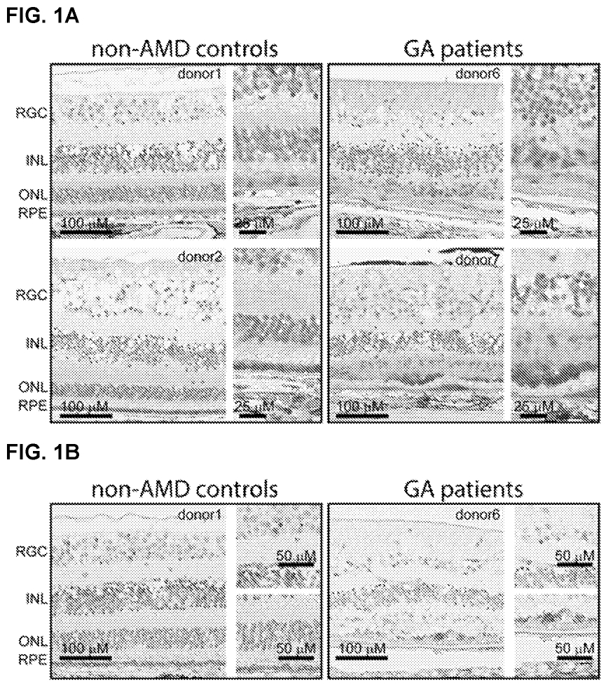

The Cpf1 CRISPR systems of the present disclosure comprise i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA (see solid triangle arrows in FIG. 1B). In some embodiments, guide sequences for Cpf1 must be at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems of the present disclosure differ from Cas9 in a variety of ways. First, unlike Cas9, Cpf1 does not require a separate tracrRNA for cleavage. In some embodiments, Cpf1 crRNAs can be as short as about 42-44 bases long—of which 23-25 nt is guide sequence and 19 nt is the constitutive direct repeat sequence. In contrast, the combined Cas9 tracrRNA and crRNA synthetic sequences can be about 100 bases long. In some embodiments, the present disclosure will refer to a crRNA for Cpf1 as a “guide RNA.”

Second, Cpf1 prefers a “TTN” PAM motif that is located 5′ upstream of its target. This is in contrast to the “NGG” PAM motifs located on the 3′ of the target DNA for Cas9 systems. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 bases, which create “sticky ends” (Kim et al., 2016. “Genome-wide analysis reveals specificities of Cpf1 endonucleases in human cells” published online Jun. 6, 2016). These sticky ends with ˜3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th base on the non-hybridized strand and the corresponding 23rd base on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on (Zetsche B. et al. 2015. “Cpf1 Is a Single RNA-Guided Endonuclease of a Class 2 CRISPR-Cas System” Cell 163, 759-771).

Persons skilled in the art will appreciate that the Cpf1 disclosed herein can be any variant derived or isolated from any source.

The present disclosure further envisions the use of catalytically inactivated Cpf1 mutants, as described in further detail below. Thus in some embodiments, the present disclosure teaches dCpf1 mutants. In some embodiments, the dCpf1 of the present disclosure comprises: ddCpf1 (Zhang et al. “Multiplex gene regulation by CRISPR ddCpf1” Cell Discovery 3, Article number 17018 (2017); Francisella novicida (UniProtKB-A0Q7Q2 (CPF1_FRATN)), Lachnospiraceae bacterium (UniProtKB-A0A182DWE3 (A0A182DWE3_9FIRM)), and Acidaminococcus sp. (UniProtKB-U2UMQ6 (CPF1 ACISB). In some embodiments, the dCpf1 of the present disclosure is generated by mutating the catalytic domain AsCpf1 (D908A Yamano, T., Nishimasu, H., Zetsche, B., Hirano, H., Slaymaker, I. M., Li, Y., Fedorova, I., Nakane, T., Makarova, K. S., Koonin, E. V. et al. (2016) Crystal Structure of Cpf1 in Complex with Guide RNA and Target DNA. Cell, 165, 949-962.

In some embodiments, the present disclosure teaches methods of modulating the expression of genes via CRISPRi (CRISPR interference). In some embodiments, the presently disclosed technologies utilize catalytically inactivated (i.e., nuclease-deactivated) CRISPR endonucleases that have been mutated to no longer generate double DNA stranded breaks, but which are still able to bind to DNA target sites through their corresponding guide RNAs. In some embodiments, the present disclosure refers to these catalytically inactivated CRISPR enzymes as “dead CRISPR”, or “dCRISPR” enzymes. The “dead” modifier may also be used in reference to specific CRISPR enzymes, such as dead Cas9 (dCas9), or dead Cpf1 (dCpf1).

Without wishing to be bound by any one theory, the dCRISPR enzymes of this technology may function by recruiting the catalytically inactivated dCRISPR enzyme to a target DNA sequence via a guide RNA, thereby permitting the dCRISPR enzyme to interact with the host cell's transcriptional machinery for a particular gene.

In some embodiments, The CRISPRi methods of the present disclosure utilize dCRISPR enzymes to occupy target DNA sequences necessary for transcription, thus blocking the transcription of the targeted gene (L. S. Qi et al., “Repurposing CRISPR as an RNA-Guided Platform for Sequence-Specific Control of Gene Expression.” Cell. 152, 1173-1183 (2013); see also L. A. Gilbert et al., “CRISPR-Mediated Modular RNA-Guided Regulation of Transcription in Eukaryotes.” Cell. 154, 442-451 (2013)). In other embodiments, the CRISPRi methods of the present disclosure utilize dCRISPR enzymes translationally fused, or otherwise tethered to one or more transcriptional repression domains, or alternatively utilize modified guide RNAs capable of recruiting transcriptional repression domains to the target site (e.g., tethered via aptamers, as discussed below).

In yet other embodiments, the presently disclosed invention also envisions exploiting dCRISPR enzymes and guide RNAs to recruit other regulatory factors to target DNA sites. In addition to recruiting transcriptional repressor domains, as discussed above, the dCRISPR enzymes and guide RNAs of the present disclosure can be modified so as to recruit proteins with activities ranging from DNA methylation, chromatin remodelers, ubiquitination, sumoylation. Thus, in some embodiments, the dCRISPR enzymes and guide RNAs of the present disclosure can be modified to recruit factors with methyltransferase activity, demethylase activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, deubiquitinating activity, adenylation activity, deadenylation activity, sumoylating activity, desumoylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, demyristoylation activity, cytidine deaminase activity and any combinations thereof.

The RIG-I inhibitor can comprise a RIG-I CRISPRi.

In some embodiments, the RIG-I inhibitor comprises a peptide or a nucleic acid molecule encoding the peptide, wherein the peptide is a TLR3 412Phe variant, LGP2, RNF125, RNF122, c-Cbl, A20, USP3, USP21, USP25, USP15, ARL16, ATG5-ATG12, NOD2, FAT10, SEC14L1, VP35, and 3C^{pro}, or a combination and/or a derivative thereof. In some embodiments, the RIG-I inhibitor comprises an anti-RIG antibody or fragment thereof. In some embodiments, the RIG-I inhibitor comprises a peptide that binds RIG-I. In various embodiments, the RIG-I inhibitor comprises a nucleic acid molecule encoding the peptide. In various embodiments, the nucleic acid molecule encodes a peptide sharing about or at least about 80%, 85%, 86%, 87%, 88%, 89%, 90%, 91%, 92%, 93%, 94%, 95%, 96%, 97%, 98%, or 99% identity to a TLR3 412Phe variant, LGP2, RNF125, RNF122, c-Cbl, A20, USP3, USP21, USP25, USP15, ARL16, ATG5-ATG12, NOD2, FAT10, SEC14L1, VP35, and 3C^{pro}. In some embodiments, the nucleic acid molecule encodes a fragment of a TLR3 412Phe variant, LGP2, RNF125, RNF122, c-Cbl, A20, USP3, USP21, USP25, USP15, ARL16, ATG5-ATG12, NOD2, FAT10, SEC14L1, VP35, and 3C^{pro}. In various embodiments, the fragment is about, at least about, or less than about 5, 10, 15, 20, 25, 30, 35, 40, 45, 50, 55, 60, 75, 80, 85, 90, 95, 100, 125, 150, 175, 200, 250, 300, 400, 500, 600, 700, 800, 900, or 1000 amino acids in length.

In some embodiments, the RIG-I inhibitor comprises an antibody (for example, a RIG-I antibody) or an antigen-binding fragment of a RIG-I antibody. RIG-I antibodies known in the art include, for example, RIG-I monoclonal antibody clone ABM40B5 (Cat. No. MBS668020; MyBioSource.com, San Diego, USA); RIG-I monoclonal antibody clone ABM4H29 (Cat. No. MBS668173: MyBioSource.com, San Diego, USA); RIG-I monoclonal antibody clone D-12 (Cat. No. sc-376845; Santa Cruz Biosciences, Santa Cruz, USA); RIG-I monoclonal antibody clone OT16C1 (Cat. No. LS-C174703: LifeSpan BioSciences, Seattle, USA); mouse IgG Anti-DDX58 antibody (Cat. No. 125746-50 ug; United States Biological, Salem, USA); RIG-I monoclonal antibody clone 4G1B6 (Cat No. MA5-31715; Thermo-Fisher Scientific, Waltham, USA); RIG-I monoclonal antibody clone 6C1 (Cat No. VMA00463; Bio-Rad, Hercules, USA); and RIG-I monoclonal antibody clone Alme-1 (Cat No, V LS-C344928-100; LSBio, Seattle, USA).

In some embodiments, the RIG-I inhibitor is introduced to an ocular cell using a vector. For example, in some embodiments, the RIG-I inhibitor is a polynucleotide, a peptide, a polypeptide, or a protein encoded by a nucleotide sequence of a vector. In some embodiments, the vector is a lentiviral vector. The inhibitor may be placed under the control of a strong pol III or pol 11 promoter. In some embodiments, the ocular cell is transduced, or transfected with the vector. The vector can be an expression vector. In some embodiments, the vector remains episomal. In some embodiments, the vector becomes chromosomally integrated, optionally using a CRISPR system. In some embodiments, the RIG-I inhibitor is chromosomally integrated into the genome of an ocular cell, optionally using a CRISPR system. The vector can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, Nature 290: 304-310, 1981), the promoter contained in the 3′ long terminal repeat of Rous sarcoma virus (Yamamoto et al., Cell 22: 787-797, 1980), the herpes thymidine kinase promoter (Wagner et al., Proc. Natl. Acad. Sci. U.S.A. 78: 1441-1445, 1981), the regulatory sequences of the metallothionein gene (Brinster et al, Nature 296: 39-42, 1982), etc. Any type of plasmid, cosmid, YAC or viral vector can be used to prepare the recombinant DNA construct, which can be introduced directly into the tissue site.

### Administration

In some embodiments, administration of the RIG-I inhibitor comprises contacting an ocular cell of the subject with the RIG-I inhibitor. In various embodiments, contacting the ocular cell of the subject with the RIG-I inhibitor comprises transducing or transfecting the ocular cell with the inhibitor or a nucleic acid molecule encoding the inhibitor. In some embodiments, contacting the ocular cell of the subject with the RIG-I inhibitor comprises introducing the RIG-I inhibitor or causing the RIG-I inhibitor to be transcribed or expressed within the ocular cell. In some embodiments, contacting the ocular cell of the subject with the RIG-I inhibitor comprises administering the RIG-I inhibitor to the subject and entry of the RIG-I inhibitor into the ocular cell. In some embodiments, contacting the ocular cell of the subject with the RIG-I inhibitor comprises physically contacting an outer surface or appendage of the ocular cell with the RIG-I inhibitor.

The methods of the present invention may draw upon many suitable modes of administration to deliver formulations comprising RIG-I inhibitors of the methods described herein. In some embodiments, the RIG-I inhibitor is administered as a pharmaceutical composition. In various embodiments, the present invention provides for pharmaceutical compositions comprising the RIG-I inhibitor. Delivery of the RIG-I inhibitor to affected regions of the body may be achieved either via local or systemic administration. Suitable formulations and additional carriers are described in Remington “The Science and Practice of Pharmacy” (20^{th} Ed., Lippincott Williams & Wilkins, Baltimore MD), the teachings of which are incorporated by reference in their entirety herein.

In some embodiments, the RIG-I inhibitor is administered systemically. It is envisioned that effective treatment can encompass administering the RIG-I inhibitor via oral administration, topical administration, via injection, intranasally, rectally, transdermally, via an impregnated or coated device such as an ocular insert or implant, or iontophoretically, amongst other routes of administration. In some embodiments, the invention provides a pharmaceutical composition for administration to a subject containing: (i) an effective amount of a RIG-I inhibitor; and (ii) a pharmaceutical excipient suitable for oral administration. In some embodiments, the composition further contains: (iii) an effective amount of a second therapeutic agent. For administration via injection, the pharmaceutical composition can be injected intramuscularly, intra-arterially, subcutaneously, or intravenously. A pump mechanism may be employed to administer the pharmaceutical composition over a preselected period.

In some embodiments, the RIG-I inhibitor is administered non-systemically. In some embodiments, the RIG-I inhibitor is administered locally to an eye of the subject, e.g. topically to the surface of the eye. For some embodiments of the invention it is desirable to deliver the RIG-I inhibitor locally, thus injections may be made periocularly, intraocularly, subconjunctively, retrobulbarly, or intercamerally.

In some embodiments, the RIG-I inhibitor is administered in a single dose. A single dose of a RIG-I inhibitor may also be used when it is co-administered with another substance for treatment of an acute condition.

In some embodiments, the RIG-I inhibitor (by itself or in combination with other drugs) is administered in multiple doses. In another embodiment the RIG-I inhibitor and another therapeutic substance are administered together.

Administration of the pharmaceutical compositions of the invention may continue as long as necessary. In some embodiments, a composition of the invention is administered for more than 1, 2, 3, 4, 5, 6, 7, 14, or 28 days. In some embodiments, a pharmaceutical composition of the invention is administered for less than 28, 14, 7, 6, 5, 4, 3, 2, or 1 day. In some embodiments, a pharmaceutical composition of the invention is administered chronically on an ongoing basis.

Dosing for the RIG-I inhibitor in the method of the invention may be found by routine experimentation. Exemplary doses can be about, at least about, or no more than about 10 μg to about 5 g. Daily dose range may depend on the form of the RIG-I inhibitor e.g., route of administration, as described herein.

### Formulations

The RIG-I inhibitors of the invention may be formulated as a sterile solution or suspension, in suitable vehicles, well known in the art. Suitable formulations and additional carriers are described in Remington “The Science and Practice of Pharmacy” (20^{th} Ed., Lippincott Williams & Wilkins, Baltimore MD), the teachings of which are incorporated by reference in their entirety herein.

For injectable formulations, the vehicle may be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles.

The concentration of RIG-I inhibitor may be adjusted, the pH of the solution buffered and the isotonicity adjusted to be compatible with intravenous injection, as is well known in the art.

Oral formulations can be tablets, capsules, troches, pills, wafers, chewing gums, lozenges, aqueous solutions or suspensions, oily suspensions, syrups, elixirs, or dispersible powders or granules, and the like and may be made in any way known in the art. Oral formulations may also contain sweetening, flavoring, coloring and preservative agents. Pharmaceutically acceptable excipients for tablet forms may comprise nontoxic ingredients such as inert diluents, such as calcium carbonate, sodium carbonate, lactose, calcium phosphate, or sodium phosphate, and the like.

In the case of tablets for oral use, carriers which are commonly used include lactose and corn starch, and lubricating agents such as magnesium stearate are commonly added. For oral administration in capsule form, useful carriers include lactose and corn starch. Further nonlimiting examples of carriers and excipients include milk, sugar, certain types of clay, gelatin, stearic acid or salts thereof, calcium stearate, talc, vegetable fats or oils, gums and glycols.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

When formulating compounds of the invention for oral administration, it may be desirable to utilize gastroretentive formulations to enhance absorption from the gastrointestinal (GI) tract. A formulation which is retained in the stomach for several hours may release compounds of the invention slowly and provide a sustained release that may be preferred in some embodiments of the invention. Disclosure of such gastro-retentive formulations are found in Klausner, E. A.; Lavy, E.; Barta, M.; Cserepes, E.; Friedman, M.; Hoffman, A. 2003 “Novel gastroretentive dosage forms: evaluation of gastroretentivity and its effect on levodopa in humans.” Pharm. Res. 20, 1466-73, Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J.; Bodmeier, R.; 2006 “Gastroretentive drug delivery systems” Expert Opin. Drug Deliver. 3, 217-3, and Chavanpatil, M. D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. Expandable, floating and bioadhesive techniques may be utilized to maximize absorption of the compounds of the invention.

For transdermal administration, any suitable formulation known in the art may be utilized, either as a solution, suspension, gel, powder, cream, oil, solids, dimethylsulfoxide (DMSO)-based solutions or liposomal formulation for use in a patch or other delivery system known in the art. The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For topical administration, all the formulations for topical ocular administration used in the field of ophthalmology (e.g., eye drops, inserts, eye packs, impregnated contact lenses, pump delivery systems, dimethylsulfoxide (DMSO)-based solutions suspensions, liposomes, and eye ointment) and all the formulations for external use in the fields of dermatology and otolaryngology (e.g., ointment, cream, gel, powder, salve, lotion, crystalline forms, foam, and spray) may be utilized as is known in the art. Additionally all suitable formulations for topical administration to skin and mucus membranes of the nasal passages may be utilized to deliver the compounds of the invention. The pharmaceutical compositions of the present invention may be a liposomal formulation for topical or oral administration, any of which are known in the art to be suitable for the purpose of this invention.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

Various formulations for administration to the eye can be prepared. For example, ocular solutions or eye drops may be prepared by formulating a RIG-I inhibitor in a sterile aqueous solution such as physiological saline, buffering solution, etc., or by combining powder pharmaceutical compositions to be dissolved before use. Other vehicles may be chosen, as is known in the art, including but not limited to: balance salt solution, saline solution, water soluble polyethers such as polyethylene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

The solubility of the components of the present pharmaceutical compositions may be enhanced by a surfactant or other appropriate co-solvent in the pharmaceutical composition. Such cosolvents include polysorbate 20, 60, and 80, Pluronic F68, F-84 and P-103, cyclodextrin, or other agents known to those skilled in the art. Such cosolvents may be employed at a level of from about 0.01% to 2% by weight.

The pharmaceutical composition of the invention can be formulated as a sterile unit dose type containing no preservatives.

The pharmaceutical compositions of the invention may be packaged in multidose form. Preservatives may be used to prevent microbial contamination during use. Non-limiting examples of suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In certain ophthalmic products, such preservatives may be employed at a level of from 0.004% to 0.02%. In the pharmaceutical compositions of the present application a preservative, such as benzalkonium chloride, may be employed at a level of from 0.001% to less than 0.01%, e.g. from 0.001% to 0.008%, or about 0.005% by weight. It has been found that a concentration of benzalkonium chloride of 0.005% may be sufficient to preserve pharmaceutical compositions from microbial attack.

The amount of administration and the number of administrations of the RIG-I inhibitor vary according to sex, age and body weight of patient, symptoms to be treated, desirable therapeutic effects, administration routes and period of treatment. In some embodiments, the RIG-I inhibitor is administered 1, 2, 3, 4, or 5 times to the subject. The RIG-I inhibitor may be administered several times a day per eye, or once a day.

The formulations of the invention can further include other pharmacological active ingredients as far as they do not contradict the purpose of the present invention. In a combination of plural active ingredients, their respective contents may be suitably increased or decreased in consideration of their effects and safety.

### Kits

The present invention also provides kits. The kits include the RIG-I inhibitor of the invention in suitable packaging, and written material that can include instructions for use, discussion of clinical studies, listing of side effects, and the like. The kit may further contain another therapeutic agent that is co-administered with the RIG-I inhibitor of the invention. In some embodiments, the therapeutic agent and the RIG-I inhibitor of the invention are provided as separate compositions in separate containers within the kit. In some embodiments, the therapeutic agent and the RIG-I inhibitor of the invention are provided as a single composition within a container in the kit. Suitable packaging and additional articles for use (e.g., syringes, foil wrapping to minimize exposure to air, dispensers, and the like) are known in the art and may be included in the kit.

### EXAMPLES

The present invention is also described and demonstrated by way of the following examples. The use of these and other examples anywhere in the specification is illustrative and in no way limits the scope and meaning of the invention or of any exemplified term. Likewise, the invention is not limited to any particular preferred embodiments described in the examples. Many modifications and variations of the invention may be apparent to those skilled in the art upon reading this specification, and such variations can be made without departing from the invention in spirit or in scope. The invention is to be limited only by the appended claims, including the full scope of equivalents to which those claims are entitled.

### Example 1: Type I IFN Response is Elevated in Human AMD Donor Eyes

To determine whether components of the type I IFN response signaling pathway are present in ocular tissues and investigate correlation of gene expression with disease pathology, ultra-fresh human donor eyes from GA patients and non-AMD control patients were analyzed. Retinal sections were analyzed for expression of IFN regulatory factor 3 (IRF3), which is believed to bind to IFN stimulated response elements (ISREs) to induce transcription of IFN genes (6,7). Immunohistochemistry (IHC) analysis of retinal tissue sections demonstrated that IRF3 was expressed in RPE and choroid layers in normal human donor eyes (FIG. 1A). The expression of IRF3 in these tissue layers indicates that RPE cells and cells of the choroid can produce IFNs.

RNAScope was used to further investigate which cells respond to IFNs. Briefly, in situ hybridization on formalin-fixed paraffin-embedded retinal samples was performed using the RNAscope® 2.5 LS-Reagent Kit-RED (Advanced Cell Diagnostics, Hayward, CA) with an ISG15 (NM_467748) specific probe according to the manufacturer's instructions. ISG15, an interferon-stimulated gene (ISGs) (8-10), was highly expressed in most ocular cells, including RGCs, ONL, INL and RPE cells, in GA patients (FIG. 1B). ISG15 expression was not observed in non-AMD control patient donor eyes (FIG. 1B). These results suggest that the IFN response was activated in GA patient retinal tissue but was not activated in ocular tissue of non-AMD patients. Patient information is provided in Table 1.

TABLE 1
Patient information used for IHC and RNAScope.

| | | | Macular |
|---|---|---|---|
| Patient ID | Age | Gender | Pathology |
| donor1 | 67 | M | normal |
| donor2 | 73 | M | normal |
| donor3 | 83 | F | normal |
| donor4 | 78 | M | normal |
| donor5 | 84 | M | normal |
| donor6 | 93 | F | GA |
| donor7 | 94 | F | GA |
| donor8 | 90 | M | GA |
| donor9 | 85 | F | GA |

### Example 2: RPE Cells can Both Produce and Respond to IFN

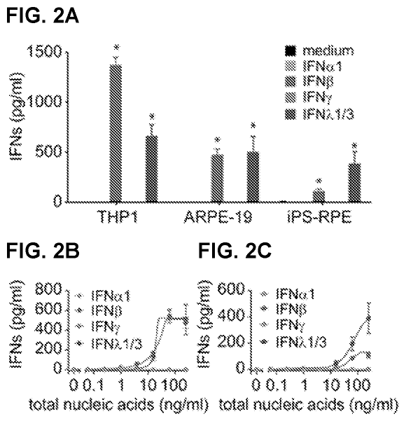

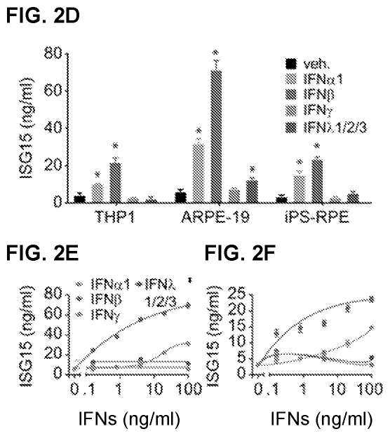

As expression of both IFN response signaling components and IFN stimulated gene mRNA expression were observed in RPE cells from human donors, RPE cells were evaluated in order to characterize the machinery of the IFN response in RPE cells and its contribution to ocular pathology. To evaluate the effect of various stimuli, ARPE-19, a spontaneously arising RPE cell line, and induced pluripotent stem (iPS)-derived RPE cells from multiple donors were used for in vitro experiments, along with THP-1 cells, a human monocytic cell line that served as a control cell line. To test the machinery of IFN signaling in these cells, both IFN production induced by transfection of nucleic acids and downstream IFN signaling markers were tested. The three major types of IFN responses were evaluated: type I (e.g., IFNα and IFNβ), type II (e.g., IFNγ) and type III (e.g., IFNλ). To characterize IFN production, transfection of a total cell nucleic acid (NA) extraction, which includes both DNA and RNA, was used to stimulate cells to avoid any bias toward individual sensors. Cells were transfected with NA at 0.25 μg/ml NA (FIG. 2A) or 0 ng/ml NA, 0.1 ng/ml NA, 1 ng/ml NA, 10 ng/ml NA or 100 ng/ml NA (FIGS. 2B and 2C). IFN expression in cell extracts was detected by ELISA using Meso Scale Discovery plates (Meso Scale Diagnostics, Rockville, MD) 24 hours after transfection. Briefly, lysates were extracted using cell lysis buffer (CST #9803), and supplemented with protease and phosphatase inhibitors. Lysates or conditioned media were measured in 384-well plates using antibody pairs for IFNβ (R&D #DY814), IFNα1 (abcam #ab215408), IFNγ (R&D #DY285B), and IFNλ1/3 (R&D #DY1598B). IFN protein levels were measured according to manufacturer instructions, and detected on a Meso Sector S 600 imager. Both IFNβ and IFNλ were detected in NA-transfected RPE cells and THP-1 cells (FIG. 2A-2C).

To test whether IFNs can induce an IFN response and, if so, which IFNs can induce an IFN response, cells were treated with different IFNs (IFNα1, IFNβ, IFNγ, or IFNλ1/2/3), and ISG15 induction was measured. Cells were treated with different IFN types at 100 ng/ml each (FIG. 2D) or 0 ng/ml IFN, 0.1 ng/ml IFN, 1 ng/ml IFN, 10 ng/ml IFN, or 100 ng/ml IFN (FIGS. 2E and 2F). ISG15 expression in was detected by ELISA 24 hours after treatment as described above, using antibody pairs for ISG15 (R&D #AF4845, #A-830). Upon treatment with different IFN types, the RPE cell lines and THP-1 cells responded to type I IFN stimulation with downstream ISG15 activation (FIG. 2D-2E). Taken together, these data show that RPE cells contained the necessary components to both produce IFN, namely IFNβ and λ, and responded to IFN stimulation, primarily IFNβ stimulation, with activation of downstream IFN signaling.

### Example 3: Identification of RIG-I as a Major Sensor of Intracellular RNA in RPE

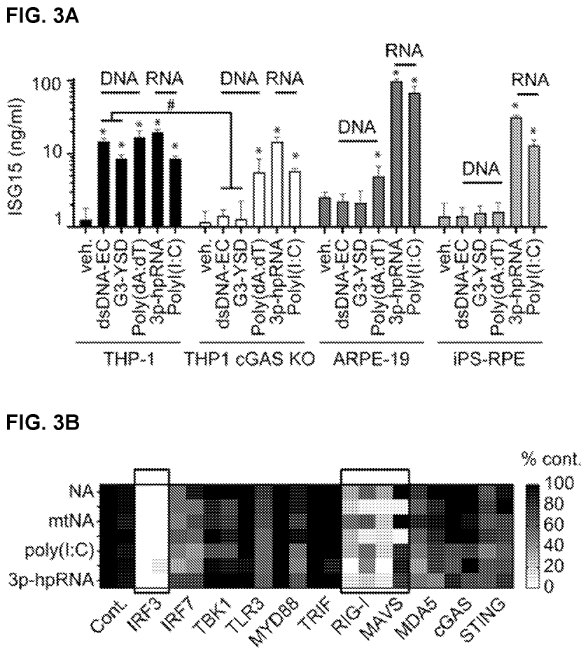

Upon showing that RPE cells can produce and respond to IFN, the mechanism of recognition of IFN-inducing stimuli was investigated. To select proper inducers for further evaluation, RPE cells and THP-1 cells were challenged with vehicle or different nucleic acids (dsDNA-EC, G3-YSD, Poly(dA:dT), 3p-hpRNA, or Poly(I:C); 0.25 μg/ml each) for 24 hours. ISG15 expression in was detected by ELISA as described above. THP-1 cells responded to multiple DNA and RNA stimuli by generating ISG15, while cGAS KO THP-1 cells (InvivoGen, San Diego, USA) showed no response to any DNA inducer, except poly(dA:dT) (FIG. 3A). Notably, poly(dA:dT) has been reported to indirectly activate IFN pathways through RIG-I via an RNA intermediary transcribed by RNA polymerase III (11,12). Surprisingly, both ARPE-19 and iPS-RPE cells responded to RNA inducers but showed no response to DNA inducers (FIG. 3A), indicating that there is little to no ability of RPE cells to sense and respond to DNA or DNA that does not activate IFN through an RNA intermediary.

In order to identify the sensors of IFN pathway activation in response to intracellular nucleic acid inducers in RPE cells, an in vitro siRNA knockdown screen targeting key nodes of the IFN pathway in ARPE-19 cells was performed. Knockdown was performed using Qiagen (Hilden, Germany) or Dharmacon (Lafayette, CO) siRNAs. Negative Control siRNAs (Cont.) were purchased from Qiagen. Transfection of siRNAs in ARPE-19 cells was carried out using Dharmafect 4 (Dharmacon). Cells were transfected with 1 pmol siRNA/96-well culture dish. Knockdown efficiency was validated using real-time reverse transcription (RT)-PCR.

Different nucleic acids, including commercially available DNA/RNA mimics (poly(I:C); 3p-hpRNA) or isolated total nucleic acid content from cells (NA) or purified mitochondria (mtNA) were tested as inducers at 0.25 μg/ml in ARPE-19 cells transfected with siRNAs. IFNβ release from ARPE-19 cells was measured using the IFNβ Human Tissue Culture Kit (Mesoscale Discovery, Rockville, MD) 24 hours after nucleic acid stimulation. Knockdown of the transcriptional regulator IRF3, was used as a positive control. IRF3 knockdown significantly inhibited nucleic acid stimulus-induced IFNβ generation (FIG. 3B). Interestingly, among multiple NA-sensing mechanisms tested, aside from IRF3, the most pronounced reduction of IFNβ production was observed in both RIG-I and MAVS knockdown groups (FIG. 3B). These results suggest that the RIG-I-MAVS-IRF3 pathway is a key pathway used by RPE cells to sense intracellular stimuli.

Further validation of the importance of RIG-I as an intracellular nucleic acid sensor was performed using CRISPR/Cas9 KO experiments in ARPE-19 cells. An ARPE-19 cell line stably expressing Cas9 (ARPE19-Cas9) was generated, along with control gRNA and RIG-I gRNA-transduced cell lines. ARPE-19 control gRNA and ARPE-19 RIG-I gRNA cells were generated by sgRNA expression in ARPE-19-Cas9 cells by lentiviral infection. The sgRNA sequence targeting DDX58/RIG-I was 5′-ACTCACCCTCCCTAAACCAG-3′ (SEQ ID NO:1). Control sgRNA sequences used were: 5′-GTAGCGAACGTGTCCGGCGT-3′ (SEQ ID NO:8), 5′-GACCGGAACGATCTCGCGTA-3′ (SEQ ID NO:9), 5′-GGCAGTCGTTCGGTTGATAT-3′ (SEQ ID NO:10), and 5′-GCTTGAGCACATACGCGAAT-3′ (SEQ ID NO: 11). ARPE19-Cas9 cells displayed a similar nucleic acid sensing profile to parental ARPE-19 cells, as determined by IFNβ production (FIG. 4A). Additionally, compared to parental cells, control gRNA-transduced cells displayed a similar nucleic acid sensing profile, but the IFNβ secretion response of RIG-I gRNA-transduced cells was completely abrogated in response to all nucleic acid species tested (FIG. 4A). All cells were stimulated with 0.25 μg/ml nucleic acid for 24 hours, after which IFNβ secretion was measured by ELISA. These results confirmed that RIG-I is a key sensor for type I IFN responsiveness in RPE cells in vitro.

In addition to the type I IFN response, nucleic acid sensing can also activate NFκB-dependent cytokine release. IL-6 secretion was measured in ARPE19-Cas9, Control gRNA, and RIG-I gRNA cell line cultures 24 hours after nucleic acid stimulation to evaluate nucleic acid-induced NFκB activation in RPE cells. All cells were stimulated with 0.25 μg/ml nucleic acid for 24 hours, after which IL-6 secretion was measured by ELISA. Interestingly, in contrast to the IFN response, poly(I:C)-induced IL-6 release was only partially reduced in ARPE-19-RIG-I KO (FIG. 4B), suggesting another RNA sensor (e.g., TLR3) played a role in poly(I:C)-induced cytokine release in RPE cells, in agreement with previous reports (13). In addition, a slight response to poly(dA:dT) was observed in ARPE-19 cells (FIG. 3A), perhaps similarly to THP-1-Dual KO-cGAS cells in which poly(dA:dT) may be recognized and initiate downstream signaling indirectly through RIG-I. This agreed with results that poly(dA:dT) did not induce downstream signaling to produce IFNβ in ARPE-19-RIG-I KO cells (FIG. 4A).

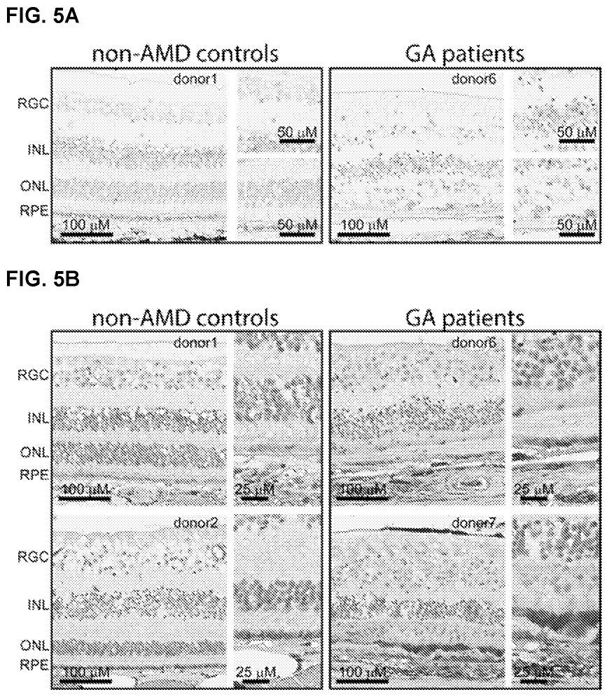

In order to investigate RIG-I expression in GA patient samples, the expression of RIG-I mRNA and protein levels were measured using RNAscope and IHC, respectively. RIG-I mRNA expression was observed to be low but detectable in retinal tissue of control human donors and relatively upregulated in GA patient eyes, notably in retinal ganglion cell (RGC), inner nuclear layer (INL), outer nuclear layer (ONL) and RPE layers (FIG. 5A), in agreement with RIG-I as a well-known mammalian ISG (14). In contrast to transcript levels, surprisingly, RIG-I protein was exclusively observed in RPE and choroid layers in both GA patients and age-matched controls (FIG. 5B), suggesting post-transcriptional mechanisms may be involved in regulating RIG-I expression in non-RPE cell types. Interestingly, RIG-I protein levels in RPE cells were still enhanced in GA patients compared to control patients (FIG. 5B), further supporting the key role of RIG-I in RPE cells.

### Example 4: cGAS Expression was not Detected in RPE Cells

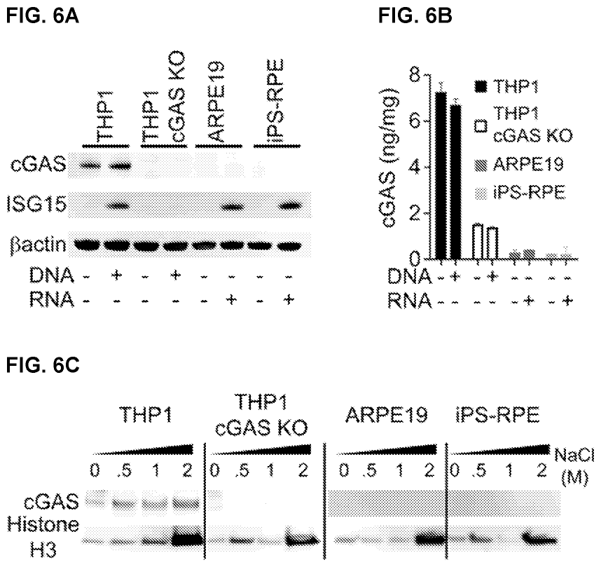

While the DNA sensor cGAS has been reported to play a major role in sensing Alu-RNA-induced RPE degeneration (4), it was a surprise that RPE cells cannot sense DNA (FIG. 3A), and that cGAS is not involved in sensing mtNA (FIG. 3B). For further confirmation, cGAS expression and function were evaluated in THP1, THP1 cGAS KO, ARPE19, and iPS-RPE cells in vitro. Expression of cGAS was measured using a cGAS antibody for western blot (CST #15102; Cell Signaling Technology; Danvers, MA, USA) that was validated using THP1-Dual KO-cGAS cells (FIG. 6A). ISG15 expression was also measured using SuperSignal™ West Femto Maximum sensitivity substrate (ThermoFisher Scientific; Waltham, MA, USA). β-actin was measured by Western blot (CST #5125). Cells were either left unstimulated or stimulated with 0.25 μg/ml of DNA or RNA. Surprisingly, no cGAS protein was detected in either ARPE-19 or iPS-RPE cells in the absence of nucleic acid stimulation (FIG. 6A). A similar pattern was observed by ELISA, where cGAS was detected in THP-1 cells but undetected in negative control THP1-Dual KO-cGAS cells and undetected in either ARPE-19 or iPS-RPE cells (FIG. 6B). For ELISA, cGAS was detected using antibody pairs (Cayman #23853, Cayman Chemical, Ann Arbor, MI, USA; and CST #66546, Cell Signaling Technology, Danvers, MA, USA). These results demonstrate that basal expression of cGAS protein was below the limit of detection by Western blot or ELISA following nucleic acid stimulation in RPE cells.

In addition, previous reports suggest that cGAS may be induced during the type I IFN response as an ISG (15,16). However, when DNA or RNA was used to stimulate THP1, THP1 cGAS KO, ARPE19, and iPS-RPE cells in vitro, no cGAS expression was observed in either ARPE-19 or iPS-RPE cells (FIGS. 6A and 6B).

Furthermore, it was reported that cGAS protein may predominantly reside in the nucleus where it interacts with, and may be sequestered by, genomic DNAs (17). To test this possibility, after extracting total nuclear protein, increasingly high doses of salt were applied to dissociate proteins and genomic DNA. Histone was used as a control to show that chromatin-bound proteins were released after high dose sodium chloride treatment, However, cGAS was not detected in ARPE19 or iPS-RPE cells following sodium chloride treatment (FIG. 6C). These observations confirmed that RPE cells did not respond to DNA stimulation (FIG. 3A), excluding the possibility that very low levels of cGAS were active in RPE.

Additionally, cGAS gene expression levels were visualized using the Single Cell Portal from Broad Institute (Cambridge, MA). cGAS was shown to be mainly expressed in myeloid and vascular cells, but not RPE. The RNA-seq data used in this analysis are described in Orozco, L. D., et al. “Integration of eQTL and a Single-Cell Atlas in the Human Eye Identifies Causal Genes for Age-Related Macular Degeneration,” Cell Reports, 30: 1246-1259.e6 (2020).

### Example 5: IFN Response Alters RPE Barrier Function Via RIG-I and not cGAS

In order to evaluate the effects of IFNs on RPE barrier function, transepithelial resistance (TER) was measured in 3D cultures of iPS-RPE cells. Briefly, barrier function of RPE cells was assessed by monitoring TER every 15 minutes by means of a cellZscope 2 (NanoAnalytics GmbH, Münster, Germany). The resistance values for individual monolayers at specific times (Ω cm²) were determined, subtracted for background resistance produced by the blank filter and culture medium (as 0%), and normalized to baseline resistance prior to stimulation (as 100%).

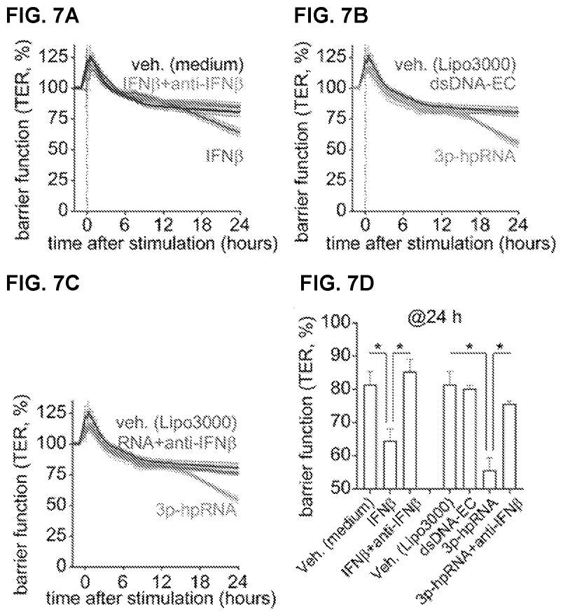

Barrier function was impaired when cells were treated with IFNβ, while co-treatment with an anti-IFNβ antibody was able to prevent the IFNβ-induced impairment (FIGS. 7A and 7D). In addition, transfection of RNA, but not DNA, resulted in impairment of TER (FIGS. 7B and 7D). Additionally, co-treatment with an anti-IFNβ antibody could prevent RNA-induced impairment or TER (FIGS. 7C and 7D). These results further support findings that the IFN response in RPE cells was induced through sensors of RNA but not DNA.

### Example 6: RNAseq Data Analysis

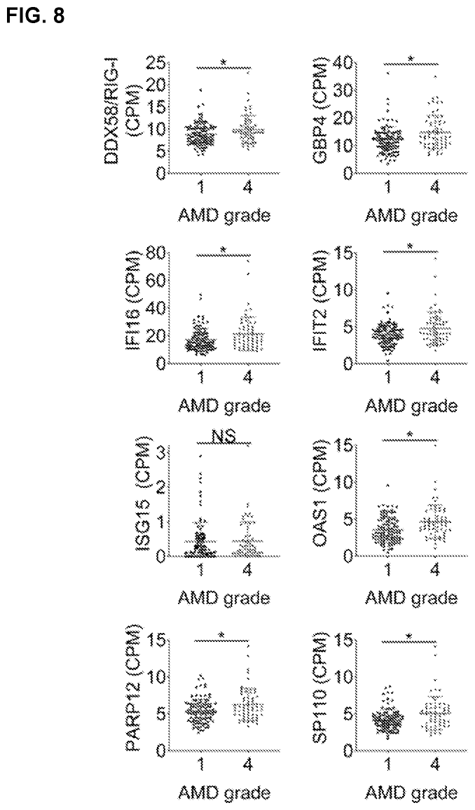

An analysis of a recently published RNAseq dataset (23) showed higher ISGs in late stage of AMD (FIG. 8). Specifically, levels of RIG-I, GBP4, IF116, IFIT2, ISG15, OAS1, PARP12, and SP110 were analyzed in AMD grade 1 and 4 patients. The significant increase in ISGs in tissue of later stage AMD patients further indicates that increased type I IFN response occurs in GA patients.

### Materials and Methods

The following materials and methods were used, unless described otherwise in a specific Example.

### Human Samples

Ultra-fresh (<6 h post-mortem) human donor eyes including geographic atrophy (GA) and non-AMD control patients were collected from Lions Eye Bank.

### Immunohistochemistry (IHC) and RNAScope

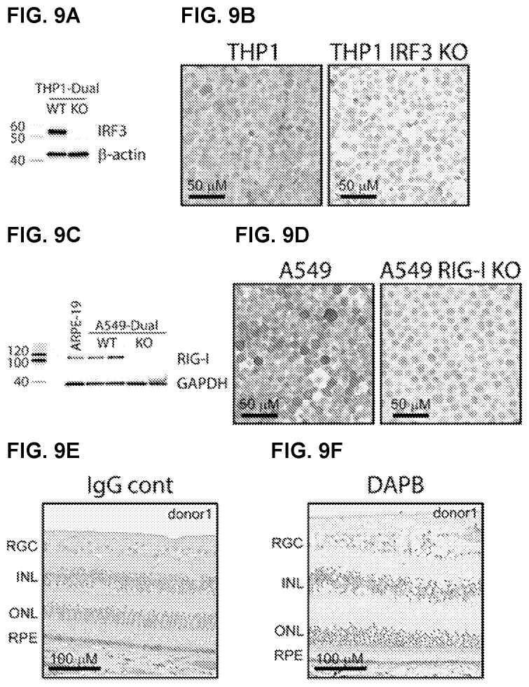

For IHC, eyes were fixed in 10% neutral buffered formalin for 2 days. Eyes were embedded in paraffin wax, serially sectioned at a thickness of 5 μm through the optic nerve, and processed for antibody staining using Leica Bond RX according to manufacturer's instructions. 20× and 40× images were taken by Aperio AT2 scanner. The specificity of IRF3 (Abcam-ab68481, Abcam, Cambridge, United Kingdom) and RIG-I (LsBIO LS-C344928, Lifespan Biosciences, Seattle, WA) antibodies were validated using THP-1-Dual and THP-1-Dual KO-IRF3 cells, A549-Dual and A549-Dual KO-RIG-I cells (InvivoGen, San Diego, CA; FIGS. 9A-D), respectively. IgG antibody was used as a negative control (FIG. 9E).

For RNAscope, in situ hybridization on formalin-fixed paraffin-embedded retinal samples was performed using the RNAscope® 2.5 LS-Reagent Kit-RED (Advanced Cell Diagnostics, Hayward, CA) with ISG15 (NM_467748) or RIG-I (NM_550268) specific probes according to the manufacturer's instructions. Probes for PPIB and DapB (Advanced Cell Diagnostics) were used as positive (data not shown) and negative controls (FIG. 9F), respectively.

### Cell Cultures

ARPE-19 was purchased from ATCC (Manassas, VA; CRL-2302). ARPE-19 was cultured in DMEM/F12 (Gibco Life Technologies, Carlsbad, CA) supplemented with 10% heat-inactivated fetal calf serum (FBS) (Sigma-Aldrich, St. Louis, MO) and 1% penicillin/streptomycin (Gibco BRL, Grand Island, NY) at 37° C. and 5% CO₂. Cells were cultured at least 3 weeks post-seeding to form mature monolayers unless otherwise specified.

iCell-RPE, an iPS-derived RPE cell line, was purchased from FUJIFILM Cellular Dynamics (Madison, WI), and represented in the figures. iPS-RPE results were further validated with at least 2-3 different donors. Cells were cultured in Lonza RtEGM medium (Lonza, Basel, Switzerland) at 37° C. and 5% CO₂. iPS-RPE cells were cultured on tissue culture treated plates, or on Falcon transmembrane inserts for polarization (Corning, Corning, NY; 353095). Cells were cultured at least 3 weeks post-seeding to form mature monolayers.

THP1-Dual, THP1-Dual KO-IRF3 and THP-1-Dual KO-cGAS cells, and A549-Dual and A549-Dual KO-RIG-I cells were purchased from InvivoGen, and cells were prepared and cultured following the manufacturer's protocols. KO cell lines were validated by western blot (FIGS. 9A and 9C).

ARPE-19 cells stably expressing the Cas9 gene (ARPE19-Cas9) were generated by lentiviral transduction of ARPE-19 cells. Virus was harvested from supernatants of HEK-293T cells (ATCC) cotransfected with the in-house lentiviral vector pNGx_LV_c010 (39) and Lentiviral Packaging Plasmid Mix (Cellecta, Mountain View, CA; #CPCP-K2A) using TransIT-293 Transfection Reagent (MirusBio, Madison, WI). ARPE19-Cas9 cells were selected with 125 μg/mL HygromycinB (Invitrogen, Carlsbad, CA) and Cas9 expression was verified by western blot using Anti-FLAG primary antibody (Sigma-Aldrich #F1804). ARPE-19 RIG-I KO cells were generated by sgRNA expression in ARPE-19-Cas9 cells by lentiviral infection using the in-house lentiviral vector pNGx_LV_g003 (39). The sgRNA sequence targeting DDX58/RIG-I is 5′-ACTCACCCTCCCTAAACCAG-3′ (SEQ ID NO: 1). Control sgRNA sequences were:

(SEQ ID NO: 8)
5′ - GTAGCGAACGTGTCCGGCGT - 3′,
(SEQ ID NO: 9)
5′ - GACCGGAACGATCTCGCGTA - 3′,
(SEQ ID NO: 10)
5′ - GGCAGTCGTTCGGTTGATAT - 3′,
and
(SEQ ID NO: 11)
5′ - GCTTGAGCACATACGCGAAT - 3′.

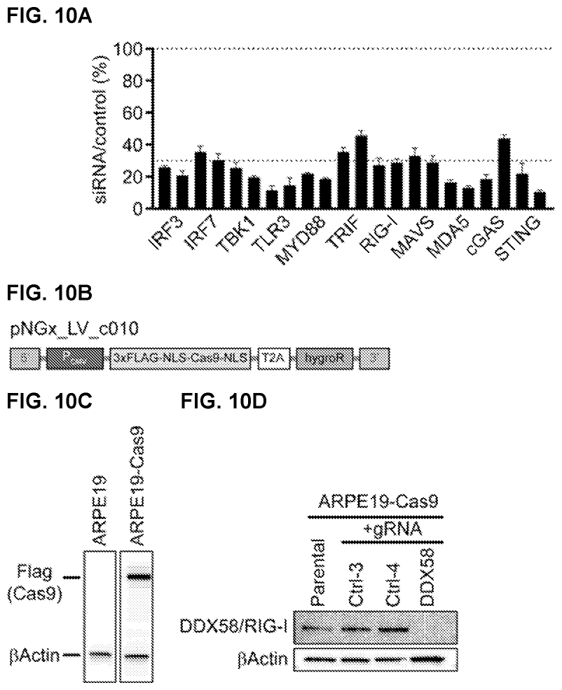

Knockout was verified by western blot (FIG. 10D).

### Reagents

IFNα, IFNβ, IFNγ, IFNλ1/2/3 were purchased from R&D Systems (Minneapolis, MN). dsDNA-EC, YSD, polydA:dT, poly(I:C)-LMW, 3p-hpRNA were purchased from InvivoGen. Total nucleic acids (NA) were isolated from ARPE-19 using Nucleic Acid Isolation Kits (ThermoFisher Scientific, Waltham, MA) according to manufacturer instructions. Mitochondrial-enriched total nucleic acid (mtNA) was prepared by isolating ARPE-19 mitochondria using Mitochondria Isolation Kit for Mammalian Cells (ThermoFisher Scientific) and then isolating nucleic acids from the mitochondrial preparation using DNeasy Blood and Tissue Kit (Qiagen). Anti-IFNβ antibody (InvivoGen) was applied to neutralize IFNβ.

### Western Blot and Enzyme-Linked Immunosorbent Assay (ELISA)

Culture supernatants were harvested, and whole-cell lysates were extracted using RIPA cell lysis buffer (Cell Signaling Technology, Danvers, MA) supplemented with protease and phosphatase inhibitors (ThermoFisher Scientific) according to the manufacturer's protocol. All samples were stored at −80° C. before use.

For western blot, samples (30 μg of protein) were dissolved in sample buffer (Invitrogen) and boiled for 5 min at 100° C. The sample was then separated by Criterion™ TGX™ precast gel electrophoresis and electrotransferred onto nitrocellulose membranes (Bio-Rad, Hercules, CA). The MagicMark™ XP Western Protein Standard (Invitrogen) was used as a molecular weight indicator. The membrane was blocked for 1 h using 5% milk in Tris-buffered saline and Tween 20 (TBST). Following three washes with TBST, the membrane was incubated overnight with primary antibodies purchased from Cell Signaling Technology, including cGAS (CST #15102), IRF3 (CST #11904), RIG-I (CST #3743), Histone H3 (CST #4499), β-actin (CST #5125) and GAPDH (CST #8884), at 1:1000 dilution. After three washes with TBST, the membrane was incubated with a secondary antibody (anti-rabbit IgG-HRP-linked antibody, at 1:1000 dilution; Cell Signaling Technology) for 1 h. The immuno-reactive proteins were detected using SuperSignal™ West Femto Maximum sensitivity substrate (ThermoFisher Scientific) and imaged on FluorChem M (ProteinSimple, San Jose, CA).

For ELISA, assays were run using Meso Scale Discovery plates (Meso Scale Diagnostics, Rockville, MD). Lysates were extracted using cell lysis buffer (CST #9803), and supplemented with protease and phosphatase inhibitors. Lysates or conditioned media were measured in 384-well plates using antibody pairs for ISG15 (R&D #AF4845, #A-830), IFNβ (R&D #DY814), IFNα1 (abcam #ab215408), IFNγ (R&D #DY285B), IFNλ1/3 (R&D #DY1598B), and cGAS (Cayman #23853, CST #66546). Protein levels were measured according to manufacturer instructions, and detected on a Meso Sector S 600 imager.

### RNA Interference

RNA interference against key nodes of the type I IFN pathway was performed using Qiagen (Hilden, Germany) or Dharmacon (Lafayette, CO) small interfering RNAs (siRNAs). The AllStars and Negative Control siRNAs were purchased from Qiagen and used as negative controls. Transfection of siRNAs in ARPE-19 cells was carried out using Dharmafect 4 (Dharmacon) according to the instructions of the manufacturer. Cells were transfected with 1 pmol/96-well culture dish for IFNβ release measurement by IFNβ Human Tissue Culture Kit (Mesoscale Discovery, Rockville, MD). The knockdown efficiency was validated using real-time reverse transcription (RT)-PCR (FIG. 10A).

### Real-Time RT-PCR

Messenger RNAs were isolated from ARPE-19 cells treated with indicated siRNAs using TurboCapture 96 mRNA Kit (Qiagen) and RNA was reverse transcribed using High-Capacity cDNA Reverse Transcription Kit (Applied Biosystems, Foster City, CA). Real-Time PCR was performed using FAM-labeled Taqman probes targeting genes of interest and a VIC-labeled Taqman probe targeting β-actin as control (Applied Biosystems). Reactions were run with Taqman Universal PCR Master Mix on the ViiA7 system (Applied Biosystems) according to the instructions of the manufacturer.

### Transepithelial Resistance (TER)

Barrier function of RPE cells was assessed by monitoring TER every 15 minutes by means of a cellZscope 2 (NanoAnalytics GmbH, Munster, Germany). The resistance values for individual monolayers at specific times (Ω cm²) were determined, subtracted for background resistance produced by the blank filter and culture medium (as 0%), and normalized to baseline resistance prior to stimulation (as 100%).

### Data Analysis

Protein levels measured by ELISA were presented as absolute amount, and gene levels by qPCR were normalized by β-actin. Three independent experiments with triplicates within each experiment were performed, and values (mean±S.D.) were presented. Two groups were compared using Student's t-test. Multiple comparisons were made using analysis of variance followed by a post hoc Newman-Keuls test. Differences were considered significant at p<0.05.

### REFERENCES

1. Wong, W. L., Su, X., Li, X., Cheung, C. M., Klein, R., Cheng, C. Y., and Wong, T. Y. (2014) Global prevalence of age-related macular degeneration and disease burden projection for 2020 and 2040: a systematic review and meta-analysis. Lancet Glob Health 2, e106-116
2. Kauppinen, A., Paterno, J. J., Blasiak, J., Salminen, A., and Kaarniranta, K. (2016) Inflammation and its role in age-related macular degeneration. Cell Mol Life Sci 73, 1765-1786
3. Knickelbein, J. E., Chan, C. C., Sen, H. N., Ferris, F. L., and Nussenblatt, R. B. (2015) Inflammatory Mechanisms of Age-related Macular Degeneration. Int Ophthalmol Clin 55, 63-78
4. Kerur, N., Fukuda, S., Banerjee, D., Kim, Y., Fu, D., Apicella, I., Varshney, A., Yasuma, R., Fowler, B. J., Baghdasaryan, E., Marion, K. M., Huang, X., Yasuma, T., Hirano, Y., Serbulea, V., Ambati, M., Ambati, V. L., Kajiwara, Y., Ambati, K., Hirahara, S., Bastos-Carvalho, A., Ogura, Y., Terasaki, H., Oshika, T., Kim, K. B., Hinton, D. R., Leitinger, N., Cambier, J. C., Buxbaum, J. D., Kenney, M. C., Jazwinski, S. M., Nagai, H., Hara, I., West, A. P., Fitzgerald, K. A., Sadda, S. R., Gelfand, B. D., and Ambati, J. (2018) cGAS drives noncanonical-inflammasome activation in age-related macular degeneration. Nat Med 24, 50-61
5. Mustafi, D., Maeda, T., Kohno, H., Nadeau, J. H., and Palczewski, K. (2012) Inflammatory priming predisposes mice to age-related retinal degeneration. J Clin Invest 122, 2989-3001
6. Honda, K., Takaoka, A., and Taniguchi, T. (2006) Type I interferon [corrected] gene induction by the interferon regulatory factor family of transcription factors. Immunity 25, 349-360
7. Tamura, T., Yanai, H., Savitsky, D., and Taniguchi, T. (2008) The IRF family transcription factors in immunity and oncogenesis. Annu Rev Immunol 26, 535-584
8. Perng, Y. C., and Lenschow, D. J. (2018) ISG15 in antiviral immunity and beyond. Nat Rev Microbiol 16, 423-439
9. Der, S. D., Zhou, A., Williams, B. R., and Silverman, R. H. (1998) Identification of genes differentially regulated by interferon alpha, beta, or gamma using oligonucleotide arrays. Proc Natl Acad Sci USA 95, 15623-15628
10. Loeb, K. R., and Haas, A. L. (1992) The interferon-inducible 15-kDa ubiquitin homolog conjugates to intracellular proteins. J Biol Chem 267, 7806-7813
11. Ablasser, A., Bauernfeind, F., Hartmann, G., Latz, E., Fitzgerald, K. A., and Hornung, V. (2009) RIG-I-dependent sensing of poly(dA:dT) through the induction of an RNA polymerase III-transcribed RNA intermediate. Nat Immunol 10, 1065-1072
12. Chiu, Y. H., Macmillan, J. B., and Chen, Z. J. (2009) RNA polymerase III detects cytosolic DNA and induces type I interferons through the RIG-I pathway. Cell 138, 576-591
13. Wornle, M., Merkle, M., Wolf, A., Ribeiro, A., Himmelein, S., Kernt, M., Kampik, A., and Eibl-Lindner, K. H. (2011) Inhibition of TLR3-mediated proinflammatory effects by Alkylphosphocholines in human retinal pigment epithelial cells. Invest Ophthalmol Vis Sci 52, 6536-6544
14. Shaw, A. E., Hughes, J., Gu, Q., Behdenna, A., Singer, J. B., Dennis, T., Orton, R. J., Varela, M., Gifford, R. J., Wilson, S. J., and Palmarini, M. (2017) Fundamental properties of the mammalian innate immune system revealed by multispecies comparison of type I interferon responses. PLoS Biol 15, e2004086
15. Ma, F., Li, B., Liu, S. Y., lyer, S. S., Yu, Y., Wu, A., and Cheng, G. (2015) Positive feedback regulation of type I IFN production by the IFN-inducible DNA sensor cGAS. J Immunol 194, 1545-1554
16. Xiong, M., Wang, S., Wang, Y. Y., and Ran, Y. (2018) The Regulation of cGAS. Virol Sin 33, 117-124
17. Volkman, H. E., Cambier, S., Gray, E. E., and Stetson, D. B. (2019) Tight nuclear tethering of cGAS is essential for preventing autoreactivity. Elife 8
18. Li, H., Ice, J. A., Lessard, C. J., and Sivils, K. L. (2013) Interferons in Sjogren's Syndrome: Genes, Mechanisms, and Effects. Front Immunol 4, 290
19. Reder, A. T., and Feng, X. (2013) Aberrant Type I Interferon Regulation in Autoimmunity: Opposite Directions in MS and SLE, Shaped by Evolution and Body Ecology. Front Immunol 4, 281
20. Mavragani, C. P., Niewold, T. B., Chatzigeorgiou, A., Danielides, S., Thomas, D., Kirou, K. A., Kamper, E., Kaltsas, G., and Crow, M. K. (2013) Increased serum type I interferon activity in organ-specific autoimmune disorders: clinical, imaging, and serological associations. Front Immunol 4, 238
21. Du, M., Wang, X., Mao, X., Yang, L., Huang, K., Zhang, F., Wang, Y., Luo, X., Wang, C., Peng, J., Liang, M., Huang, D., and Huang, K. (2019) Absence of Interferon Regulatory Factor 1 Protects Against Atherosclerosis in Apolipoprotein E-Deficient Mice. Theranostics 9, 4688-4703
22. Taylor, J. M., Minter, M. R., Newman, A. G., Zhang, M., Adlard, P. A., and Crack, P. J. (2014) Type-1 interferon signaling mediates neuro-inflammatory events in models of Alzheimer's disease. Neurobiol Aging 35, 1012-1023
23. Ratnapriya, R., Sosina, O. A., Starostik, M. R., Kwicklis, M., Kapphahn, R. J., Fritsche, L. G., Walton, A., Arvanitis, M., Gieser, L., Pietraszkiewicz, A., Montezuma, S. R., Chew, E. Y., Battle, A., Abecasis, G. R., Ferrington, D. A., Chatterjee, N., and Swaroop, A. (2019) Retinal transcriptome and eQTL analyses identify genes associated with age-related macular degeneration. Nat Genet 51, 606-610
24. Kumar, M. V., Nagineni, C. N., Chin, M. S., Hooks, J. J., and Detrick, B. (2004) Innate immunity in the retina: Toll-like receptor (TLR) signaling in human retinal pigment epithelial cells. J Neuroimmunol 153, 7-15
25. Patel, A. K., and Hackam, A. S. (2013) Toll-like receptor 3 (TLR3) protects retinal pigmented epithelium (RPE) cells from oxidative stress through a STAT3-dependent mechanism. Mol Immunol 54, 122-131
26. Yang, Z., Stratton, C., Francis, P. J., Kleinman, M. E., Tan, P. L., Gibbs, D., Tong, Z., Chen, H., Constantine, R., Yang, X., Chen, Y., Zeng, J., Davey, L., Ma, X., Hau, V. S., Wang, C., Harmon, J., Buehler, J., Pearson, E., Patel, S., Kaminoh, Y., Watkins, S., Luo, L., Zabriskie, N. A., Bernstein, P. S., Cho, W., Schwager, A., Hinton, D. R., Klein, M. L., Hamon, S. C., Simmons, E., Yu, B., Campochiaro, B., Sunness, J. S., Campochiaro, P., Jorde, L., Parmigiani, G., Zack, D. J., Katsanis, N., Ambati, J., and Zhang, K. (2008) Toll-like receptor 3 and geographic atrophy in age-related macular degeneration. N Engl J Med 359, 1456-1463
27. Dhir, A., Dhir, S., Borowski, L. S., Jimenez, L., Teitell, M., Rotig, A., Crow, Y. J., Rice, G. I., Duffy, D., Tamby, C., Nojima, T., Munnich, A., Schiff, M., de Almeida, C. R., Rehwinkel, J., Dziembowski, A., Szczesny, R. J., and Proudfoot, N. J. (2018) Mitochondrial double-stranded RNA triggers antiviral signalling in humans. Nature 560, 238-242
28. West, A. P., Khoury-Hanold, W., Staron, M., Tal, M. C., Pineda, C. M., Lang, S. M., Bestwick, M., Duguay, B. A., Raimundo, N., MacDuff, D. A., Kaech, S. M., Smiley, J. R., Means, R. E., Iwasaki, A., and Shadel, G. S. (2015) Mitochondrial DNA stress primes the antiviral innate immune response. Nature 520, 553-557
29. Bianchi, E., Scarinci, F., Ripandelli, G., Feher, J., Pacella, E., Magliulo, G., Gabrieli, C. B., Plateroti, R., Plateroti, P., Mignini, F., and Artico, M. (2013) Retinal pigment epithelium, age-related macular degeneration and neurotrophic keratouveitis. Int J Mol Med 31, 232-242
30. Orozco, L. D., Chen, H. H., Cox, C., Katschke, K. J., Jr., Arceo, R., Espiritu, C., Caplazi, P., Nghiem, S. S., Chen, Y. J., Modrusan, Z., Dressen, A., Goldstein, L. D., Clarke, C., Bhangale, T., Yaspan, B., Jeanne, M., Townsend, M. J., van Lookeren Campagne, M., and Hackney, J. A. (2020) Integration of eQTL and a Single-Cell Atlas in the Human Eye Identifies Causal Genes for Age-Related Macular Degeneration. Cell Rep 30, 1246-1259 e1246
31. Chawla-Sarkar, M., Lindner, D. J., Liu, Y. F., Williams, B. R., Sen, G. C., Silverman, R. H., and Borden, E. C. (2003) Apoptosis and interferons: role of interferon-stimulated genes as mediators of apoptosis. Apoptosis 8, 237-249
32. Li, Y., Guo, X., Hu, C., Du, Y., Guo, C., Di, W., Zhao, W., Huang, G., Li, C., Lu, Q., Lai, R., Xu, T., and Qi, X. (2018) Type I IFN operates pyroptosis and necroptosis during multidrug-resistant A. baumannii infection. Cell Death Differ 25, 1304-1318
33. Stawowczyk, M., Van Scoy, S., Kumar, K. P., and Reich, N. C. (2011) The interferon stimulated gene 54 promotes apoptosis. J Biol Chem 286, 7257-7266
34. Thapa, R. J., Nogusa, S., Chen, P., Maki, J. L., Lerro, A., Andrake, M., Rall, G. F., Degterev, A., and Balachandran, S. (2013) Interferon-induced RIP1/RIP3-mediated necrosis requires PKR and is licensed by FADD and caspases. Proc Natl Acad Sci USA 110, E3109-3118
35. Sarhan, J., Liu, B. C., Muendlein, H. I., Weindel, C. G., Smirnova, I., Tang, A. Y., Ilyukha, V., Sorokin, M., Buzdin, A., Fitzgerald, K. A., and Poltorak, A. (2019) Constitutive interferon signaling maintains critical threshold of MLKL expression to license necroptosis. Cell Death Differ 26, 332-347
36. Baechler, E. C., Batliwalla, F. M., Karypis, G., Gaffney, P. M., Ortmann, W. A., Espe, K. J., Shark, K. B., Grande, W. J., Hughes, K. M., Kapur, V., Gregersen, P. K., and Behrens, T. W. (2003) Interferon-inducible gene expression signature in peripheral blood cells of patients with severe lupus. Proc Natl Acad Sci USA 100, 2610-2615
37. Bennett, L., Palucka, A. K., Arce, E., Cantrell, V., Borvak, J., Banchereau, J., and Pascual, V. (2003) Interferon and granulopoiesis signatures in systemic lupus erythematosus blood. J Exp Med 197, 711-723
38. Xiong, W., Wu, D. M., Xue, Y., Wang, S. K., Chung, M. J., Ji, X., Rana, P., Zhao, S. R., Mai, S., and Cepko, C. L. (2019) AAV cis-regulatory sequences are correlated with ocular toxicity. Proc Natl Acad Sci USA 116, 5785-5794
39. DeJesus, R., Moretti, F., McAllister, G., Wang, Z., Bergman, P., Liu, S., Frias, E., Alford, J., Reece-Hoyes, J. S., Lindeman, A., Kelliher, J., Russ, C., Knehr, J., Carbone, W., Beibel, M., Roma, G., Ng, A., Tallarico, J. A., Porter, J. A., Xavier, R. J., Mickanin, C., Murphy, L. O., Hoffman, G. R., and Nyfeler, B. (2016) Functional CRISPR screening identifies the ufmylation pathway as a regulator of SQSTM1/p62. Elife 5

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

Patents, patent applications, publications, product descriptions, and protocols are cited throughout this application, the disclosures of which are incorporated herein by reference in their entireties for all purposes.

What is claimed is:

1. A method for treating a retinal degenerative disease or condition in a subject in need thereof, the method comprising administering to the subject a therapeutically effective amount of a retinoic acid-inducible gene I (RIG-I) inhibitor.

2. The method of claim 1, wherein the retinal degenerative disease or condition is age-related macular degeneration (AMD).

3. The method of claim 1 or 2, wherein the AMD is geographic atrophy (GA).

4. A method for reducing the progression of GA lesion enlargement in a subject, the method comprising administering to the subject a therapeutically effective amount of a RIG-I inhibitor.

5. A method for improving vision in a subject, the method comprising administering to the subject a therapeutically effective amount of a RIG-I inhibitor.

6. A method for inhibiting RPE degeneration in an eye of a subject, the method comprising administering to the subject a therapeutically effective amount of a RIG-I inhibitor.

7. A method for inhibiting transepithelial resistance impairment of RPE in an eye of a subject, the method comprising administering to the subject a therapeutically effective amount of a RIG-I inhibitor.

8. The method of any one of claims 1 to 7, wherein the RIG-I inhibitor comprises a small molecule chemical compound, an antibody, an antigen-binding fragment of a RIG-I specific antibody, a nucleic acid molecule, a peptide, or a derivative thereof.

9. The method of claim 1 to 8, wherein the RIG-I inhibitor comprises a siRNA, a miRNA, a shRNA, a ribozyme, a morpholino, an antisense RNA, a triple helix forming molecule, or a sgRNA.

10. The method of any one of claims 1 to 9, wherein the RIG-I inhibitor comprises a RIG-I CRISPRi system.

11. The method of any one of claims 1 to 9, wherein the RIG-I inhibitor comprises a RIG-I CRISPR-Cas9 system.

12. The method of claim 11, wherein the RIG-I CRISPR-Cas9 system comprises an sgRNA comprising the sequence 5′-ACTCACCCTCCCTAAACCAG-3′ (SEQ ID NO:1), or a derivative thereof.

13. The method of any one of claims 1 to 8, wherein the RIG-I inhibitor comprises a RIG-I siRNA.

14. The method of claim 13, wherein, the RIG-I siRNA comprises a nucleotide sequence selected from the group consisting of:
(SEQ ID NO: 2)
5′ - AAGGGAACGATTCCATCACTA - 3′,
(SEQ ID NO: 3)
5′ - TTCTACAGATTTGCTCTACTA - 3′,
(SEQ ID NO: 4)
5′ - CTCCTCCTACCCGGCTTTAAA - 3′,
and
(SEQ ID NO: 5)
5′ - CAGAATTATCCCAACCGATAT - 3′.

15. The method of any one of claims 1 to 8, wherein the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof.

16. The method of any one of claims 1 to 8, wherein the RIG-I inhibitor comprises the peptide sequence of SEQ ID NO:6 (GNRDTLWHLFNTLQRRPGWVEYFI), or a derivative thereof; or a nucleic acid molecule encoding the peptide of SEQ ID NO:6, or a derivative thereof.

17. The method of any one of claims 1 to 16, wherein the RIG-I inhibitor further comprises a vector.

18. The method of claim 17, wherein the vector is a viral vector, optionally a lentiviral vector.

19. The method of any one of claims 1 to 8, wherein the small molecule compound is selected from the group consisting of: 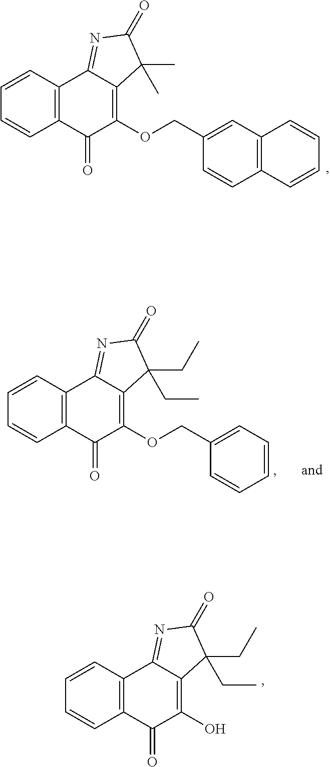 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]
or a pharmaceutically acceptable salt thereof.

20. The method of any one of claims 1 to 19, wherein said administering is oral, transdermal, topical, parenteral, by injection, or by inhalation.

21. The method of claim 20, wherein said administering is locally to an eye of the subject.

22. The method of claim 21, wherein said administering is subconjunctival, intravitreal, periocular, retrobulbar, or intracameral.

23. The method of any one of claims 1 to 22, wherein said administering comprises contacting an ocular cell of the subject with the RIG-I inhibitor.

24. The method of any one of claims 1 to 22, wherein said administering is effective to reduce expression of an interferon stimulated gene (ISG) in an ocular cell of the subject.

25. The method of claim 24, wherein said ISG is ISG15.

26. The method of any of claims 1 to 22, wherein said administering is effective to reduce RIG-I protein level in an ocular cell of the subject.

27. The method of any one of claims 1 to 22, wherein said administering is effective to reduce RIG-I mRNA level in an ocular cell of the subject.

28. The method of any one of claims 1 to 22, wherein said administering is effective to reduce an IFN response in an ocular cell of the subject.

29. The method of claim 28, wherein said IFN response is a type I IFN response.

30. The method of any one of claims 1 to 22, wherein said administering is effective to reduce expression and/or secretion of a type I interferon in an ocular cell of the subject.

31. The method of claim 30, wherein the type I interferon is IFNβ.

32. The method of any one of claims 1 to 22, wherein said administering is effective to inhibit nucleic acid-induced inflammation in an ocular cell of the subject.

33. The method of any one of claims 23 to 32, wherein the ocular cell is a retinal ganglion (RGC) cell, a cell in the inner nuclear layer (INL), a cell in the outer nuclear layer (ONL), a retinal pigmented epithelium (RPE) cell, a cell in the choroidal layer, or a combination thereof.

34. The method of any one of claims 23 to 32, wherein the ocular cell is an RPE cell.

35. The method of claim 33, wherein the cell in the INL is a horizontal cell, a bipolar cell, an amacrine cell, an interplexiform neuron, a Müller cell, or a combination thereof.

36. The method of claim 33, wherein the cell in the ONL is a cone cell, a rod cell, a photoreceptor cell, or a combination thereof.

37. The method of any one of claims 1 to 36, wherein said administering is effective to inhibit RPE degeneration.

38. The method of any one of claims 1 to 37, wherein said administering is effective to inhibit transepithelial resistance impairment of the RPE.

39. The method of any one of claims 1 to 38, wherein the subject is a human.

40. A method for inhibiting an IFN response in an ocular cell, the method comprising contacting the cell with a RIG-I inhibitor.

41. A method for inhibiting expression and/or secretion of a type I IFN in an ocular cell, the method comprising contacting the cell with a RIG-I inhibitor.

42. The method of claim 41, wherein the type I interferon is IFNβ.

43. A method for inhibiting nucleic acid-induced inflammation in an ocular cell, the method comprising contacting the cell with a RIG-I inhibitor.

44. A method for inhibiting RIG-I expression in a cell, the method comprising: contacting the cell with a RIG-I inhibitor.

45. The method of any one of claims 40 to 44, wherein the ocular cell is a retinal ganglion (RGC) cell, a cell in the inner nuclear layer (INL), a cell in the outer nuclear layer (ONL), a retinal pigmented epithelium (RPE) cell, a cell in the choroidal layer, or a combination thereof.

46. The method of any one of claims 40 to 44, wherein the ocular cell is a retinal ganglion (RGC) cell, a cell in the INL, a cell in the ONL, a RPE cell, a cell in the choroidal layer, or a combination thereof.

47. The method of any one of claims 40 to 44, wherein the ocular cell is an RPE cell, a cell in the choroidal layer, or a combination thereof.

48. The method of any one of claims 40 to 44, wherein the ocular cell is an RPE cell.

49. The method of claim 46, wherein the cell in the INL is a horizontal cell, a bipolar cell, an amacrine cell, an interplexiform neuron, a Müller cell, or a combination thereof.

50. The method of claim 46, wherein the cell in the ONL is a cone cell, a rod cell, a photoreceptor cell, or a combination thereof.

51. The method of any one of claims 40 to 50, wherein the RIG-I inhibitor comprises a small molecule chemical compound, an antibody, an antigen-binding fragment of a RIG-I antibody, a nucleic acid molecule, a peptide, or a derivative thereof.

52. The method of any one of claims 40 to 51, wherein the RIG-I inhibitor comprises a siRNA, a miRNA, a shRNA, a ribozyme, a morpholino, an antisense RNA, a triple helix forming molecule, or a sgRNA.

53. The method of any one of claims 40 to 52, wherein the RIG-I inhibitor comprises a RIG-I CRISPRi system.

54. The method of any one of claims 40 to 52, wherein the RIG-I inhibitor comprises a RIG-I CRISPR-Cas9 system.

55. The method of claim 54, wherein the RIG-I CRISPR-Cas9 system comprises an sgRNA comprising the sequence 5′-ACTCACCCTCCCTAAACCAG-3′ (SEQ ID NO:1), or a derivative thereof.

56. The method of any one of claims 40 to 52, wherein the RIG-I inhibitor comprises a RIG-I siRNA.

57. The method of claim 56, wherein, the RIG-I siRNA comprises a nucleotide sequence selected from the group consisting of:
(SEQ ID NO: 2)
5′ - AAGGGAACGATTCCATCACTA - 3′,
(SEQ ID NO: 3)
5′ - TTCTACAGATTTGCTCTACTA - 3′,
(SEQ ID NO: 4)
5′ - CTCCTCCTACCCGGCTTTAAA - 3′,
and
(SEQ ID NO: 5)
5′ - CAGAATTATCCCAACCGATAT - 3′

58. The method of any one of claims 40 to 51, wherein the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof.

59. The method of any one of claims 40 to 51, wherein the RIG-I inhibitor comprises the peptide sequence of SEQ ID NO:6 (GNRDTLWHLFNTLQRRPGWVEYFI), or a derivative thereof; or a nucleic acid molecule encoding the peptide of SEQ ID NO:6, or a derivative thereof.

60. The method of any one of claims 40 to 59, wherein the RIG-I inhibitor further comprises a vector.

61. The method of claim 60, wherein the vector is a viral vector, optionally a lentiviral vector.

62. The method of any one of claims 40 to 51, wherein the small molecule compound is selected from the group consisting of: 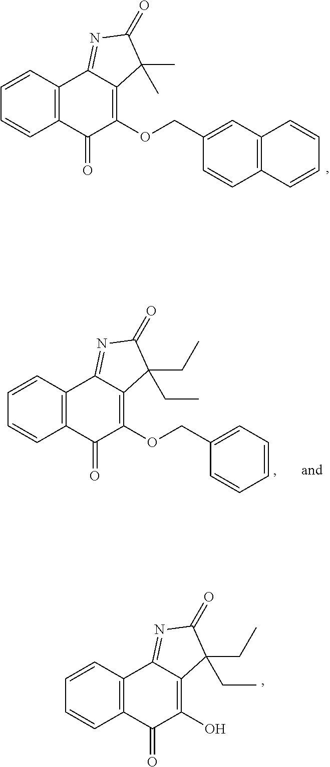 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]
or a pharmaceutically acceptable salt thereof.

63. The method of any one of claims 40 to 62, wherein said contacting is effective to reduce expression of an interferon stimulated gene (ISG) in the ocular cell.

64. The method of claim 63, wherein said ISG is ISG15.

65. The method of any one of claims 40 to 64, wherein said contacting is effective to reduce RIG-I protein levels in the ocular cell.

66. The method of any one of claims 40 to 65, wherein said contacting is effective to reduce RIG-I mRNA levels in the ocular cell.

67. The method of any one of claims 40 to 66, wherein said contacting is effective to reduce IFN response signaling in the ocular cell.

68. The method of claim 67, wherein said IFN response signaling comprises expression of a type I interferon.

69. The method of any one of claims 40 to 68, wherein said contacting is effective to reduce expression and/or secretion of a type I interferon in the ocular cell.

70. The method of claim 68 or 69, wherein the type I interferon is IFNβ.

71. The method of any one of claims 40 to 70, wherein said contacting is effective to inhibit nucleic acid-induced inflammation signaling in the ocular cell.

72. A method for reducing toxicity of an ocular gene therapy in a subject, the method comprising administering to the subject a therapeutically effective amount of a RIG-I inhibitor.

73. The method of claim 72, wherein the RIG-I inhibitor comprises a small molecule chemical compound, an antibody, an antigen-binding fragment of a RIG-I antibody, a nucleic acid molecule, a peptide, or a derivative thereof.

74. The method of claim 72 or claim 73, wherein the RIG-I inhibitor comprises a siRNA, a miRNA, a shRNA, a ribozyme, a morpholino, an antisense RNA, a triple helix forming molecule, or a sgRNA.

75. The method of any one of claims 72 to 74, wherein the RIG-I inhibitor comprises a RIG-I CRISPRi system.

76. The method of any one of claims 72 to 74, wherein the RIG-I inhibitor comprises a RIG-I CRISPR-Cas9 system.

77. The method of claim 76, wherein the RIG-I CRISPR-Cas9 system comprises an sgRNA comprising the sequence 5′-ACTCACCCTCCCTAAACCAG-3′ (SEQ ID NO: 1), or a derivative thereof.

78. The method of any one of claims 72 to 74, wherein the RIG-I inhibitor comprises a RIG-I siRNA.

79. The method of claim 78, wherein, the RIG-I siRNA comprises a nucleotide sequence selected from the group consisting of:
(SEQ ID NO: 2)
5′ - AAGGGAACGATTCCATCACTA - 3′,
(SEQ ID NO: 3)
5′ - TTCTACAGATTTGCTCTACTA - 3′,
(SEQ ID NO: 4)
5′ - CTCCTCCTACCCGGCTTTAAA - 3′,
and
(SEQ ID NO: 5)
5′ - CAGAATTATCCCAACCGATAT - 3′

80. The method of claim 72 or 73, wherein the RIG-I inhibitor comprises a peptide or a nucleic acid molecule encoding the peptide, wherein the peptide is selected from the group consisting of: LGP2, RNF125, RNF122, c-Cbl, A20, USP3, USP21, USP25, USP15, ARL16, ATG5-ATG12, NOD2, FAT10, SEC14L1, VP35, and 3C^{pro}, or a derivative thereof.

81. The method of claim 72 or 73, wherein the RIG-I inhibitor comprises the peptide sequence of SEQ ID NO:6 (GNRDTLWHLFNTLQRRPGWVEYFI), or a derivative thereof; or a nucleic acid molecule encoding the peptide of SEQ ID NO:6, or a derivative thereof.

82. The method of any one of claims 72 to 81, wherein the RIG-I inhibitor further comprises a vector.

83. The method of claim 82, wherein the vector is a viral vector, optionally a lentiviral vector.

84. The method of claim 72 or 73, wherein the small molecule compound is selected from the group consisting of: 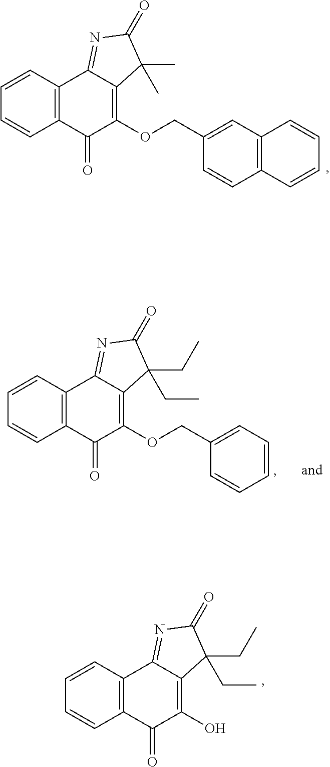 [SMILES: CC1(C)C(=O)N=C2C1=C(OCc1ccc3ccccc3c1)C(=O)c1ccccc12 ; CCC1(CC)C(=O)N=C2C1=C(OCc1ccccc1)C(=O)c1ccccc12 ; CCC1(CC)C(=O)N=C2C1=C(O)C(=O)c1ccccc12]
or a pharmaceutically acceptable salt thereof.

85. The method of any one of claims 72 to 84, wherein said administering is oral, transdermal, topical, parenteral, by injection, or by inhalation.

86. The method of claim 85, wherein said administering is locally to an eye of the subject.

87. The method of claim 86, wherein said administering is subconjunctival, intravitreal, periocular, retrobulbar, or intracameral.

88. The method of any one of claims 72 to 87, wherein said administering comprises contacting an ocular cell of the subject with the RIG-I inhibitor.

89. The method of any one of claims 72 to 87, wherein said administering is effective to reduce expression of an interferon stimulated gene (ISG) in an ocular cell of the subject.

90. The method of claim 89, wherein said ISG is ISG15.

91. The method of any one of claims 72 to 87, wherein said administering is effective to reduce RIG-I protein level in an ocular cell of the subject.

92. The method of any one of claims 72 to 87, wherein said administering is effective to reduce RIG-I mRNA level in an ocular cell of the subject.

93. The method of any one of claims 72 to 87, wherein said administering is effective to reduce an IFN response in an ocular cell of the subject.

94. The method of claim 93, wherein said IFN response is a type I IFN response.

95. The method of any one of claims 72 to 87, wherein said administering is effective to reduce expression and/or secretion of a type I interferon in an ocular cell of the subject.

96. The method of claim 95, wherein the type I interferon is IFNβ.

97. The method of any one of claims 72 to 87, wherein said administering is effective to inhibit nucleic acid-induced inflammation in an ocular cell of the subject.

98. The method of any one of claims 88 to 97, wherein the ocular cell is a retinal ganglion (RGC) cell, a cell in the INL, a cell in the ONL, a RPE cell, a cell in the choroidal layer, or a combination thereof.

99. The method of any one of claims 88 to 97, wherein the ocular cell is an RPE cell or a cell in the choroidal layer.

100. The method of any one of claims 88 to 97, wherein the ocular cell is an RPE cell.

101. The method of claim 98, wherein the cell in the INL is a horizontal cell, a bipolar cell, an amacrine cell, an interplexiform neuron, or a Müller cell.

102. The method of claim 98, wherein the cell in the ONL is a cone cell, a rod cell, or a photoreceptor cell.

103. The method of any one of claims 72 to 102, wherein the subject is a human.

104. The method of any one of claims 72 to 103, wherein the gene therapy comprises administering an AAV gene therapy vector to the subject.

105. The method of any one of claims 72 to 104, wherein the gene therapy comprises subretinally injecting an AAV gene therapy vector.

106. The method of any one of claims 72 to 105, wherein said administering is effective to treat macular degeneration (AMD) associated with the gene therapy toxicity.

107. The method of any one of claims 72 to 105, wherein said administering is effective to treat geographic atrophy (GA) associated with the gene therapy toxicity.


SEQUENCE LISTING (13 sequences)

SEQ ID NO: 1 - DNA, 20 bp, Artificial Sequence
  source: /note=“Description of Artificial Sequence Synthetic oligonucleotide”
actcaccctccctaaaccag

SEQ ID NO: 2 - DNA, 21 bp, Artificial Sequence
  source: /note=“Description of Artificial Sequence Synthetic oligonucleotide”
aagggaacgattccatcacta

SEQ ID NO: 3 - DNA, 21 bp, Artificial Sequence
  source: /note=“Description of Artificial Sequence Synthetic oligonucleotide”
ttctacagatttgctctacta

SEQ ID NO: 4 - DNA, 21 bp, Artificial Sequence
  source: /note=“Description of Artificial Sequence Synthetic oligonucleotide”
ctcctcctacccggctttaaa

SEQ ID NO: 5 - DNA, 21 bp, Artificial Sequence
  source: /note=“Description of Artificial Sequence Synthetic oligonucleotide”
cagaattatcccaaccgatat

SEQ ID NO: 6 - protein, 24 aa, Homo sapiens
GNRDTLWHLFNTLQRRPGWVEYFI

SEQ ID NO: 7 - protein, 540 aa, Homo sapiens
MPFAEDKTYKYICRNFSNFCNVDVVEILPYLPCLTARDQDRLRATCTLSGNRDTLWHLFNTLQRRPGWVEYFIAALRGCELVDLADEVASVYQSYQPRTSDRPPDPLEPPSLPAERPGPPTPAAAHSIPYNSCREKEPSYPMPVQETQAPESPGENSEQALQTLSPRAIPRNPDGGPLESSSDLAALSPLTSSGHQEQDTELGSTHTAGATSSLTPSRGPVSPSVSFQPLARSTPRASRLPGPTGSVVSTGTSFSSSSPGLASAGAAEGKQGAESDQAEPIICSSGAEAPANSLPSKVPTTLMPVNTVALKVPANPASVSTVPSKLPTSSKPPGAVPSNALTNPAPSKLPINSTRAGMVPSKVPTSMVLTKVSASTVPTDGSSRNEETPAAPTPAGATGGSSAWLDSSSENRGLGSELSKPGVLASQVDSPFSGCFEDLAISASTSLGMGPCHGPEENEYKSEGTFGIHVAENPSIQLLEGNPGPPADPDGGPRPQADRKFQEREVPCHRPSPGALWLQVAVTGVLVVTLLVVLYRRRLH

SEQ ID NO: 8 - DNA, 20 bp, Artificial Sequence
  source: /note=“Description of Artificial Sequence Synthetic oligonucleotide”
gtagcgaacgtgtccggcgt

SEQ ID NO: 9 - DNA, 20 bp, Artificial Sequence
  source: /note=“Description of Artificial Sequence Synthetic oligonucleotide”
gaccggaacgatctcgcgta

SEQ ID NO: 10 - DNA, 20 bp, Artificial Sequence
  source: /note=“Description of Artificial Sequence Synthetic oligonucleotide”
ggcagtcgttcggttgatat

SEQ ID NO: 11 - DNA, 20 bp, Artificial Sequence
  source: /note=“Description of Artificial Sequence Synthetic oligonucleotide”
gcttgagcacatacgcgaat

SEQ ID NO: 12 - DNA, 4628 bp, Homo sapiens
gaacgtagctagctgcaagcagaggccggcatgaccaccgagcagcgacgcagcctgcaagccttccaggattatatccggaagaccctggaccctacctacatcctgagctacatggccccctggtttagggaggaagaggtgcagtatattcaggctgagaaaaacaacaagggcccaatggaggctgccacactttttctcaagttcctgttggagctccaggaggaaggctggttccgtggctttttggatgccctagaccatgcaggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatgatatcaggtcctcaatcttcagctacagggaatgagtaactttgagtggagaagaaacaaacatagtgggtataatcatggatcgcttgtacccctgtgaaaatatattttttaaaaatatctttagcagtttgtactatattatatatgcaaagcacaaatgagtgaatcacagcactgagtattttgtaggccaacagagctcatagtacttgggaaaaattaaaaagcctcatttctagccttctttttagagtcaactgccaacaaacacacagtaatcactctgtacacactgggatagatgaatgaatggaatgttgggaatttttatctccctttgtctccttaacctactgtaaactggcttttgcccttaacaatctactgaaattgttcttttgaaggttaccagtgactctggttgccaaatccactgggcacttcttaaccttctatttgacctctgcgcatttggccctgttgagcactcttcttgaagctctccctgggcttctctctcttctagttctattctagtctttttttattgagtcctcctctttgctgatcccttccaagggttcaatatatatacatgtatatactgtacatatgtatatgtaactaatatacatacatacaggtatgtatatgtaatggttatatgtactcatgttcctggtgtagcaacgtgtggtatggctacacagagaacatgagaacataaagccatttttatgcttactactaaaagctgtccactgtagagttgctgtatgtagcaatgtgtatccactctacagtggtcagcttttagtagagagcataaaaatgataaaatacttcttgaaaacttagtttactatacatcttgccctattaatatgttctcttaacgtgtgccattgttctctttgaccattttcctataatgatgttgatgttcaacacctggactgaatgtctgttctcagatcccttggatgttacagatgaggcagtctgactgtcctttctacttgaaagattagaatatgtatccaaatggcattcacgtgtcacttagcaaggtttgctgatgcttcaaagagcttagtttgcggtttcctggacgtggaaacaagtatctgagttccctggagatcaacgggatgaggtgttacagctgcctccctcttcatgcaatctggtgagcagtggtgcaggcggggagccagagaaacttgccagttatataacttctctttggcttttcttcatctgtaaaacaaggataatactgaactgtaagggttagtggagagtttttaattaaaagaatgtgtgaaaagtacatgacacagtagttgcttgataatagttactagtagtagtattcttactaagacccaatacaaatggattatttaaaccaagtttatgagttggttttttttcattttctatttgtattttattaagagtgtcttttcttatgtgattttttttaattgctatttgatatggtttggctatatgtccccacccaaatctcatcttgaattataatccccatgtgtcaagggagggacctgacgggaggtgattggatcacgggggcagttgtccccatgctgttcttgggatagtgagttagttctcatgagatctgatggttttataagtgtttgacaattcctcctttacacacactctctctctcatctgctgccatgtaagacttgcctgcttccccttctgccatgattgtaagtttcctgaggcctcctcagccatgtggaactgtgaatctattaagcctcttttctttataaatga

SEQ ID NO: 13 - protein, 925 aa, Homo sapiens
MTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRNYQLELALPAMKGKNTIICAPTGCGKTFVSLLICEHHLKKFPQGQKGKVVFFANQIPVYEQQKSVFSKYFERHGYRVTGISGATAENVPVEQIVENNDIIILTPQILVNNLKKGTIPSLSIFTLMIFDECHNTSKQHPYNMIMFNYLDQKLGGSSGPLPQVIGLTASVGVGDAKNTDEALDYICKLCASLDASVIATVKHNLEELEQVVYKPQKFFRKVESRISDKFKYIIAQLMRDTESLAKRICKDLENLSQIQNREFGTQKYEQWIVTVQKACMVFQMPDKDEESRICKALFLYTSHLRKYNDALIISEHARMKDALDYLKDFFSNVRAAGFDEIEQDLTQRFEEKLQELESVSRDPSNENPKLEDLCFILQEEYHLNPETITILFVKTRALVDALKNWIEGNPKLSFLKPGILTGRGKTNQNTGMTLPAQKCILDAFKASGDHNILIATSVADEGIDIAQCNLVILYEYVGNVIKMIQTRGRGRARGSKCFLLTSNAGVIEKEQINMYKEKMMNDSILRLQTWDEAVFREKILHIQTHEKFIRDSQEKPKPVPDKENKKLLCRKCKALACYTADVRVIEECHYTVLGDAFKECFVSRPHPKPKQFSSFEKRAKIFCARQNCSHDWGIHVKYKTFEIPVIKIESFVVEDIATGVQTLYSKWKDFHFEKIPFDPAEMSK